(19) United States
(12) Patent Application Publication
(10) Pub. No.: US 2023/0080690 A1
(43) Pub. Date: Mar. 16, 2023

(54) CARTILAGE DAMAGE TREATMENT MATERIAL UTILIZING BONE MARROW FLUID

(71) Applicants: National University Corporation Hokkaido University, Sapporo-shi, Hokkaido (JP); Mochida Pharmaceutical Co., Ltd., Shinjuku-ku, Tokyo (JP)

(72) Inventors: Norimasa IWASAKI, Sapporo-shi, Hokkaido (JP); Tomohiro ONODERA, Sapporo-shi, Hokkaido (JP); Atsushi URITA, Sapporo-shi, Hokkaido (JP); Ryo JO, Sapporo-shi, Hokkaido (JP)

(73) Assignees: National University Corporation Hokkaido University, Sapporo-shi, Hokkaido (JP); Mochida Pharmaceutical Co., Ltd., Shinjuku-ku, Tokyo (JP)

(21) Appl. No.: 17/792,076

(22) PCT Filed: Jan. 15, 2021
(86) PCT No.: PCT/JP2021/001162

§ 371 Date: Jul. 11, 2022

(30) Foreign Application Priority Data
Jan. 17, 2020 (JP) 2020-005679

(51) Int. Cl. A61K 35/28; A61P 19/02; A61K 31/734

(52) U.S. Cl. CPC A61K 35/28; A61P 19/02; A61K 31/734

(57) ABSTRACT

Provided is a composition for treating cartilage injury lesion that is combined with a concentrated bone marrow aspirate and applied to a cartilage injury lesion, that has flowability when applied to the cartilage injury lesion, and that contains a monovalent metal salt of alginic acid. Thereby, a novel composition for treating cartilage injury lesion that can be used to restore and/or regenerate cartilage tissue is provided.

### TECHNICAL FIELD

The present invention relates to a composition and a method and the like for cartilage injury treatment.

### BACKGROUND ART

The joint is surrounded by an articular capsule, the inner surface of which is lined with a synovial membrane. The joint cavity between the bones making up the joint is filled with a synovial fluid secreted by the synovial membrane. The surfaces of the bones at the joint are covered with articular cartilage. The articular cartilage has a 4-layer structure comprising a superficial layer, an intermediate layer, a deep layer and a calcified layer. Bone tissue called subchondral bone lies below the deepest calcified layer, and the calcified layer is tightly connected to the subchondral bone.

Articular cartilage is hyaline cartilage that is composed of a small number of cells, collagenous extracellular matrix, abundant proteoglycans and water. Since vascular and neural networks are present in the bone and the bone has self-repairing ability, even when a bone is fractured, the fractured part is often adequately repaired. Articular cartilage, however, lacks vascular and neural networks. Therefore, the cartilage has almost no self-repairing ability, and cartilage defects are not adequately repaired particularly when large cartilage defects have formed. Even if the parts are repaired, the parts are remodeled with fibrocartilage having different mechanical properties from hyaline cartilage. Thus, when cartilage defects form, the defects may cause joint pain and loss of joint function, often leading to osteoarthritis. Furthermore, cartilage defects may develop over a broad area as symptoms progress from the initial stages of osteoarthritis, in which the surface of articular cartilage begins to wear due to aging or overuse of joints.

In this context, attempts have been made to utilize natural polymers such as collagen, chitosan, agarose and alginic acid in regenerative medicine for articular cartilage and in the treatment of joint disease. For example, the use of alginate in the regeneration of articular and other cartilage and in the treatment of joint disease has been proposed (see for example PTL 1 to 4 and NPL 1). Efforts have also been made to use alginate in combination with cells, and, for example, favorable regeneration of hyaline cartilage almost equivalent to normal cartilage has been reported when bone marrow mesenchymal stem cells were embedded in an alginate and applied to damaged cartilage parts (see for example PTL 1 and NPL 1). Progress has also been made in using growth factors and cytokines together with alginate in cartilage regeneration treatment (PTL 3). In another report, combined treatment with an alginate and bone marrow stimulation (BMS) promoted cartilage repair, but no significant effect was reported on repair of subchondral bone (NPL 2).

Another treatment method that has been reported for articular cartilage defects is intra-articular injection of autologous concentrated bone marrow fluid (NPL 3 to 5). Being a liquid, concentrated bone marrow fluid has fluidity and flows with the movement of the joints without adhering even when the fluid is transplanted directly into a bone marrow defect. Regarding treatment by transplantation of concentrated bone marrow fluid, concentrated bone marrow fluid has also been combined and transplanted in combination with various biological material in order to achieve favorable adhesion and fixation at the defect site (NPL 6, 7). However, in the case of combined transplantation of concentrated bone marrow fluid, techniques such as shape adjustment and fixing to the defect site are necessary in order to match the biological materials to the shape of the cartilage defect during surgery.

### CITATION LIST

### Patent Literature

[PTL 1] WO 2008/102855

[PTL 2] WO 2009/054181

[PTL 3] WO 2013/027854

[PTL 4] WO 2017/175229

### Non Patent Literature

[NPL 1] Igarashi et al., J Biomed Mater Res Part A (2012) 100A, 180-187

[NPL 2] Baba et al., Tissue Eng Part C Methods (2015) 21 (12), 1263-73

[NPL 3] Kristin et al., Recent Adv Arthroplast (2018) 2 (2), 75-81

[NPL 4] Kim et al., Eur J Orthop Surg Traumatol (2014) 24, 1505-1511

[NPL 5] Shapiro et al., Am J Sports Med (2017) 45, 82-90

[NPL 6] Gobbi et al., Cartilage (2011) 2 (3), 286-299

[NPL 7] Cavallo et al., J Biomed Mater Res A (2013) 101 (6), 1559-70

### SUMMARY OF INVENTION

### Technical Problem

Under these circumstances, there is demand for a novel composition for treating cartilage injury that can be used for repairing and/or regenerating injury to cartilage tissue.

### Solution to Problem

As a result of diligent studies, the inventors perfected the present invention after discovering that a relatively large area of damaged cartilage tissue could be repaired and/or regenerated by applying a concentrated bone marrow aspirate to a damaged cartilage site in combination with a monovalent metal salt of alginic acid.

That is, the following are provided here.

[1-1] A composition for treating cartilage injury that is applied to a cartilage injury lesion of a subject in combination with a concentrated bone marrow aspirate, and that has fluidity when applied to the cartilage injury lesion, and that contains a monovalent metal salt of alginic acid.
[1-2] The composition according to [1-1] above, wherein the concentrated bone marrow aspirate is derived from the subject.
[1-3] The composition according to [1-1] or [1-2] above, wherein the application is performed according to either (1) or (2) below.

(1) a concentrated bone marrow aspirate is mixed with a composition containing a monovalent metal salt of alginic acid, and the resulting mixture is applied to a cartilage injury lesion of a subject.

(2) a concentrated bone marrow aspirate and a composition containing a monovalent metal salt of alginic acid are each applied to a cartilage injury lesion of a subject rather than being mixed before application to the cartilage injury lesion.

[1-4] The composition according to any one of [1-1] to [1-3] above, wherein the number of bone marrow mesenchymal stem cells in the concentrated bone marrow aspirate is from 1×10² to 1×10⁷ cells/mL.
[1-5] The composition according to any one of [1-1] to [1-4] above, wherein the cartilage injury is accompanied by injury to the subchondral bone.
[1-6] The composition according to any one of [1-1] to [1-5] above, wherein the cartilage injury lesion is at least 4 cm² in size.
[1-7] The composition according to any one of [1-1] to [1-6] above, wherein the cartilage injury lesion is articular cartilage.
[1-8] The composition according to any one of [1-1] to [1-7] above, wherein an apparent viscosity of the composition having fluidity before being combined with the concentrated bone marrow aspirate is from 500 mPa·s to 10,000 mPa·s as measured with a cone plate viscometer (sensor 35/1) with a measurement temperature of 20° C. for the measurement, a rotational speed of 0.5 rpm and a reading time of 2.5 minutes and using the average of the values taken during the period from 0.5 to 2.5 minutes after the start of measurement.
[1-9] The composition according to any one of [1-1] to [1-8] above, wherein the monovalent metal salt of alginic acid has a weight-average molecular weight (absolute molecular weight) of at least 80,000 as measured by a GPC-MALS method.
[1-10] The composition according to any one of [1-1] to [1-9] above, wherein a concentration of the monovalent metal salt of alginic acid in the composition before being combined with the concentrated bone marrow aspirate is from 0.5 w/w % to 5 w/w %.
[1-11] The composition according to any one of [1-1] to [1-10] above, wherein the composition is used by bringing a curing agent into contact with at least part of a surface of the composition after the composition is applied to the cartilage injury lesion.
[1-12] The composition according to any one of [1-1] to [1-11] above, wherein the composition does not contain the curing agent in an amount that cures the composition before being applied to the cartilage injury lesion of the subject.
[1-13] The composition according to [1-11] or [1-12] above, wherein the curing agent is a divalent or higher metal ion compound.
[1-14] The composition according to any one of [1-1] to [1-13] above, wherein the monovalent metal salt of alginic acid is a low-endotoxin monovalent metal salt of alginic acid.
[1-15] The composition according to any one of [1-1] to [1-14] above, wherein the composition is used to treat a cartilage-related disease.
[1-16] The composition according to [1-15] above, wherein the cartilage-related disease is at least one cartilage-related disease selected from the group consisting of osteoarthritis, traumatic cartilage defect, peripheral traumatic cartilage defect, traumatic cartilage injury, osteonecrosis, osteochondritis dissecans, subchondral bone defect, degeneration of cartilage and/or subchondral bone, and misalignment in joints.
[1-17] The composition according to any one of [1-1] to [1-16] above, wherein the composition is used for at least one selected from the group consisting of repairing cartilage tissue and/or subchondral bone, regenerating cartilage tissue and/or subchondral bone, suppressing progress of degeneration in cartilage tissue and/or subchondral bone, regenerating hyaline cartilage, alleviating pain, alleviating postoperative pain, reducing dysfunction, alleviating clinical symptoms, and preventing or suppressing the recurrence of cartilage-related disease.

[2-1] A kit for treating cartilage injury containing at least a composition according to any one of [1-1] to [1-17] above together with a curing agent.

[2-1A] The kit according to [2-1] above, wherein the kit is used by applying the composition in combination with a concentrated bone marrow aspirate to a cartilage injury lesion of a subject.
[2-2] The kit according to [2-1] or [2-1A] above, wherein the concentrated bone marrow aspirate is derived from the subject.
[2-3] The kit according to any one of [2-1] to [2-2] above, wherein the application is performed according to either (1) or (2) below.

(1) a concentrated bone marrow aspirate is mixed with a composition containing a monovalent metal salt of alginic acid, and the resulting mixture is applied to a cartilage injury lesion of a subject.

(2) a concentrated bone marrow aspirate and a composition containing a monovalent metal salt of alginic acid are each applied to a cartilage injury lesion of a subject rather than being mixed before application to the cartilage injury lesion.

[2-4] The kit according to any one of [2-1] to [2-3] above, wherein the number of bone marrow mesenchymal stem cells in the concentrated bone marrow aspirate is from 1 x 10² to 1×10⁷ cells/mL.
[2-5] The kit according to any one of [2-1] to [2-4] above, wherein the cartilage injury is accompanied by damage to the subchondral bone.
[2-6] The kit according to any one of [2-1] to [2-5] above, wherein the cartilage injury lesion is at least 4 cm² in size.
[2-7] The kit according to any one of [2-1] to [2-6] above, wherein the cartilage injury lesion is articular cartilage.
[2-8] The kit according to any one of [2-1] to [2-7] above, wherein an apparent viscosity of the composition having fluidity before being combined with the concentrated bone marrow aspirate is from 500 mPa·s to 10,000 mPa·s as measured with a cone plate viscometer (sensor 35/1) with a measurement temperature of 20° C. for the measurement, a rotational speed of 0.5 rpm and a reading time of 2.5 minutes and using the average of the values taken during the period from 0.5 to 2.5 minutes after the start of measurement.
[2-9] The kit according to any one of [2-1] to [2-8] above, wherein the monovalent metal salt of alginic acid has a weight-average molecular weight (absolute molecular weight) of at least 80,000 as measured by a GPC-MALS method.
[2-10] The kit according to any one of [2-1] to [2-9] above, wherein a concentration of the monovalent metal salt of alginic acid in the composition before being combined with the concentrated bone marrow aspirate is from 0.5 w/w % to 5 w/w %.
[2-11] The kit according to any one of [2-1] to [2-10] above, wherein the kit is used by bringing a curing agent into contact with at least part of a surface of the composition after the composition is applied to the cartilage injury lesion.
[2-12] The kit according to any one of [2-1] to [2-11] above, wherein the composition does not contain the curing agent in an amount that cures the composition before being applied to the cartilage injury lesion of the subject.
[2-13] The kit according to [2-11] or [2-12] above, wherein the curing agent is a divalent or higher metal ion compound.
[2-14] The kit according to any one of [2-1] to [2-13] above, wherein the monovalent metal salt of alginic acid is a low-endotoxin monovalent metal salt of alginic acid.
[2-15] The kit according to any one of [2-1] to [2-14] above, wherein the kit is used to treat a cartilage-related disease.
[2-16] The kit according to [2-15] above, wherein the cartilage-related disease is at least one cartilage-related disease selected from the group consisting of osteoarthritis, traumatic cartilage defect, peripheral traumatic cartilage defect, traumatic cartilage injury, osteonecrosis, osteochondritis dissecans, subchondral bone defect, degeneration of cartilage and/or subchondral bone, and misalignment in joints.
[2-17] The kit according to any one of [2-1] to [2-16] above, wherein the kit is used for at least one selected from the group consisting of repairing cartilage tissue and/or subchondral bone, regenerating cartilage tissue and/or subchondral bone, suppressing progress of degeneration in cartilage tissue and/or subchondral bone, regenerating hyaline cartilage, alleviating pain, alleviating postoperative pain, reducing dysfunction, alleviating clinical symptoms, and preventing or suppressing the recurrence of cartilage-related disease.

[3-1] A method for treating cartilage injury in a subject by applying a composition containing a monovalent metal salt of alginic acid to a cartilage injury lesion of the subject in combination with a concentrated bone marrow aspirate, wherein the composition has fluidity when applied to the cartilage injury lesion.

[3-2] The method according to [3-1] above, wherein the concentrated bone marrow aspirate is derived from the subject.
[3-3] A method according to [3-1] or [3-2] above, wherein the application is performed according to either (1) or (2) below.

(1) a concentrated bone marrow aspirate is mixed with a composition containing a monovalent metal salt of alginic acid, and the resulting mixture is applied to a cartilage injury lesion of a subject.

(2) a concentrated bone marrow aspirate and a composition containing a monovalent metal salt of alginic acid are each applied to a cartilage injury lesion of a subject rather than being mixed before application to the cartilage injury lesion.

[3-4] The method according to any one of [3-1] to [3-3] above, wherein the number of bone marrow mesenchymal stem cells in the concentrated bone marrow aspirate is from 1×10² to 1×10⁷ cells/mL.
[3-5] The method according to any one of [3-1] to [3-4] above, wherein the cartilage injury is accompanied by damage to the subchondral bone.
[3-6] The method according to any one of [3-1] to [3-5] above, wherein the cartilage injury lesion is at least 4 cm² in size.
[3-7] The method according to any one of [3-1] to [3-6] above, wherein the cartilage injury lesion is articular cartilage.
[3-8] The method according to any one of [3-1] to [3-7] above, wherein an apparent viscosity of the composition having fluidity before being combined with the concentrated bone marrow aspirate is from 500 mPa·s to 10,000 mPa·s as measured with a cone plate viscometer (sensor 35/1) with a measurement temperature of 20° C. for the measurement, a rotational speed of 0.5 rpm and a reading time of 2.5 minutes and using the average of the values taken during the period from 0.5 to 2.5 minutes after the start of measurement.
[3-9] The method according to any one of [3-1] to [3-8] above, wherein the monovalent metal salt of alginic acid has a weight-average molecular weight (absolute molecular weight) of at least 80,000 as measured by a GPC-MALS method.
[3-10] The method according to any one of [3-1] to [3-9] above, wherein a concentration of the monovalent metal salt of alginic acid in the composition before being combined with the concentrated bone marrow aspirate is from 0.5 w/w % to 5 w/w %.
[3-11] The method according to any one of [3-1] to [3-10] above, wherein a curing agent is brought into contact with at least part of a surface of the composition after the composition is applied to the cartilage injury lesion.
[3-12] The method according to any one of [3-1] to [3-11] above, wherein the composition does not contain the curing agent in an amount that cures the composition before being applied to the cartilage injury lesion of the subject.
[3-13] The method according to [3-11] or [3-12] above, wherein the curing agent is a divalent or higher metal ion compound.
[3-14] The method according to any one of [3-1] to [3-13] above, wherein the monovalent metal salt of alginic acid is a low-endotoxin monovalent metal salt of alginic acid.
[3-15] The method according to any one of [3-1] to [3-14] above, wherein the cartilage injury is a cartilage-related disease.
[3-16] The method according to [3-15] above, wherein the cartilage-related disease is at least one cartilage-related disease selected from the group consisting of osteoarthritis, traumatic cartilage defect, peripheral traumatic cartilage defect, traumatic cartilage injury, osteonecrosis, osteochondritis dissecans, subchondral bone defect, degeneration of cartilage and/or subchondral bone, and misalignment in joints.
[3-17] The method according to any one of [3-1] to [3-16] above, wherein the cartilage injury treatment includes at least one selected from the group consisting of repairing cartilage tissue and/or subchondral bone, regenerating cartilage tissue and/or subchondral bone, suppressing progress of degeneration in cartilage tissue and/or subchondral bone, regenerating hyaline cartilage, alleviating pain, alleviating postoperative pain, reducing dysfunction, alleviating clinical symptoms, and preventing or suppressing the recurrence of cartilage-related disease.

[4-1] A monovalent metal salt of alginic acid used for treating cartilage damage, which is applied to a damaged cartilage site in combination with a concentrated bone marrow aspirate and has fluidity when applied to the damaged cartilage site.

[4-2] The monovalent metal salt of alginic acid according to [4-1] above, wherein the concentrated bone marrow aspirate is derived from the subject.
[4-3] A monovalent metal salt of alginic acid according to [4-1] or [4-2] above, wherein the application is performed according to either (1) or (2) below.

(1) a concentrated bone marrow aspirate is mixed with a monovalent metal salt of alginic acid, and the resulting mixture is applied to a damaged cartilage site of a subject.

(2) a concentrated bone marrow aspirate and a monovalent metal salt of alginic acid are each applied to a cartilage injury lesion of a subject rather than being mixed before application to the cartilage injury lesion.

[4-4] The monovalent metal salt of alginic acid according to any one of [4-1] to [4-3] above, wherein the number of bone marrow mesenchymal stem cells in the concentrated bone marrow aspirate is from 1×10² to 1×10⁷ cells/mL.
[4-5] The monovalent metal salt of alginic acid according to any one of [4-1] to [4-4] above, wherein the cartilage injury is accompanied by damage to the subchondral bone.
[4-6] The monovalent metal salt of alginic acid according to any one of [4-1] to [4-5] above, wherein the cartilage injury lesion is at least 4 cm² in size.
[4-7] The monovalent metal salt of alginic acid according to any one of [4-1] to [4-6] above, wherein the cartilage injury lesion is articular cartilage.
[4-8] The monovalent metal salt of alginic acid according to any one of [4-1] to [4-7] above, wherein an apparent viscosity of the monovalent metal salt of alginic acid having fluidity before being combined with the concentrated bone marrow aspirate is from 500 mPa·s to 10,000 mPa·s as measured with a cone plate viscometer (sensor 35/1) with a measurement temperature of 20° C. for the measurement, a rotational speed of 0.5 rpm and a reading time of 2.5 minutes and using the average of the values taken during the period from 0.5 to 2.5 minutes after the start of measurement.
[4-9] The monovalent metal salt of alginic acid according to any one of [4-1] to [4-8] above, wherein the monovalent metal salt of alginic acid has a weight-average molecular weight (absolute molecular weight) of at least 80,000 as measured by a GPC-MALS method.
[4-10] The monovalent metal salt of alginic acid according to any one of [4-1] to [4-9] above, in the form of a composition in which a concentration of the monovalent metal salt of alginic acid before being combined with the concentrated bone marrow aspirate is from 0.5 w/w % to 5 w/w %.
[4-11] The monovalent metal salt of alginic acid according to any one of [4-1] to [4-10] above, wherein the monovalent metal salt of alginic acid is used by bringing a curing agent into contact with at least part of a surface of the monovalent metal salt of alginic acid after it is applied to the damaged cartilage site.
[4-12] The monovalent metal salt of alginic acid according to any one of [4-1] to [4-11] above, wherein the monovalent metal salt of alginic acid does not contain the curing agent in an amount that cures the monovalent metal salt of alginic acid before being applied to the damaged cartilage site of the subject.
[4-13] The monovalent metal salt of alginic acid according to [4-11] or [4-12] above, wherein the curing agent is a divalent or higher metal ion compound.
[4-14] The monovalent metal salt of alginic acid according to any one of [4-1] to [4-13] above, wherein the monovalent metal salt of alginic acid is a low-endotoxin monovalent metal salt of alginic acid.
[4-15] The monovalent metal salt of alginic acid according to any one of [4-1] to [4-14] above, wherein the monovalent metal salt of alginic acid is used to treat a cartilage-related disease.
[4-16] The monovalent metal salt of alginic acid according to [4-15] above, wherein the cartilage-related disease is at least one cartilage-related disease selected from the group consisting of osteoarthritis, traumatic cartilage defect, peripheral traumatic cartilage defect, traumatic cartilage injury, osteonecrosis, osteochondritis dissecans, subchondral bone defect, degeneration of cartilage and/or subchondral bone, and misalignment in joints.
[4-17] The monovalent metal salt of alginic acid according to any one of [4-1] to [4-16] above, wherein the monovalent metal salt of alginic acid is used for at least one selected from the group consisting of repairing cartilage tissue and/or subchondral bone, regenerating cartilage tissue and/or subchondral bone, suppressing progress of degeneration in cartilage tissue and/or subchondral bone, regenerating hyaline cartilage, alleviating pain, alleviating postoperative pain, reducing dysfunction, alleviating clinical symptoms, and preventing or suppressing the recurrence of cartilage-related disease.

### Effect of the Invention

Provided is a novel composition for treating cartilage injury that can be used for repairing and/or regenerating damaged cartilage tissue.

### BRIEF DESCRIPTION OF DRAWINGS

FIG. 1 shows a schematic view and a macroscopic photograph from each osteochondral defect surgery model group.

FIG. 2 shows typical macroscopic findings for each group 4 weeks (A to D) and 16 weeks (F to I) after surgery and ICRS score results (E, J). The scores are represented as average values and standard deviations for each group, with * being P<0.05, **P<0.01 and ***P<0.001.

FIG. 3 shows histological findings and Niederauer score results 4 weeks and 16 weeks after surgery. Microscopic images (×20) (A to D) and enlarged images (×100) (E to H) are shown from 4 weeks after surgery. Microscopic images (×20) (J to M) and enlarged images (×100) (N to Q) are shown from 16 weeks after surgery. The histological scores for each group 4 weeks (I) and 16 weeks (R) after surgery are represented as average values and standard deviations, with * being P<0.05, **P<0.01 and ***P<0.001. The Scalebar shows units of 1 mm.

FIG. 4 shows the results of an evaluation of collagen orientation in repaired tissue 16 weeks after surgery. A to D shows tissue sections that were subjected to anti-type II collagen immunostaining. The Scalebar shows units of 1 mm. E to P are photographs of HE stained tissue sections that were rotated by 0, 45 and 90 degrees with a polarized light microscope (PLM). Q shows average values and standard deviations for degenerative changes in repaired tissue in each group (**=P<0.01).

FIG. 5 shows the results of an evaluation of the amount of subchondral bone repair as evaluated by micro-CT 16 weeks after surgery. A to D are reconstructed 2D (Axial) and 3D-CT synthetic images for each group 4 weeks after surgery. G to J are reconstructed 2D (Axial) and 3D-CT synthetic images for each group 16 weeks after surgery. The amounts of subchondral bone repair (BV) for each group 4 weeks (F) and 16 weeks (K) after surgery are represented as average values and standard deviations (*=P<0.05, **=P<0.01 and ***=P<0.001).

FIG. 6 shows the results of an evaluation of mechanical properties 16 weeks after surgery. A is a schematic diagram of a mechanical test. The tip has a 2 mm hemispheric shape. B is an external view of the indentation tester. C shows typical press-fit deformation curves for repaired tissue as calculated from each group. D shows normalized stiffness values for repaired tissue in each group, represented as average values and standard deviations (*=P<0.05).

### DESCRIPTION OF EMBODIMENTS

The details are explained below.

### 1. Composition of Invention

Provided here is a composition that is used favorably for treating cartilage injury.

This composition is a cartilage injury treatment composition (sometimes called “the composition” in this Description) that is applied in combination with a concentrated bone marrow aspirate to a cartilage injury lesion of a subject, has fluidity when applied to the cartilage injury lesion, and contains a monovalent metal salt of alginic acid. This composition is preferably used by bringing a curing agent into contact with at least a part of the surface of the composition after it is applied to the cartilage injury lesion.

“Cartilage” is found in joints, thoracic walls, intervertebral discs and meniscus as well as tubular structures such as the larynx, airways and ears, and is classified into three types, hyaline cartilage, elastic cartilage and fibrocartilage. For example, joint cartilage is hyaline cartilage that is composed of chondrocytes, collagenous extracellular matrix, proteoglycans and water, with no blood vessels distributed in the cartilage. Hyaline cartilage is rich in type II collagen, is stained by anti-type II collagen antibodies, and is also stained red by safranin-O staining for proteoglycans among other properties.

“Cartilage injury” means a state in which cartilage is damaged due to aging, trauma or various other factors, and includes states of reduced cartilage function, such as when the characteristic viscoelasticity of cartilage (the property of compressing slowly under load and slowly returning to its original state when the load is removed) declines for some reason, causing difficulty with supporting loads even though mobility is maintained. Unless otherwise specified, “injury” in this Description includes “degeneration”.

A “cartilage defect” is a site where the cartilage layer is missing and refers to a cavity in cartilage tissue as well as the surrounding tissue forming the cavity. Cartilage defects are one form of cartilage injury, and the composition is preferably used for treating cartilage defect site.

A “injury site” in this Description means the same as a “lesion”, and the term “lesion” may be used interchangeably with the term “injury”.

In certain embodiments, a cartilage injury lesion or cartilage defect site is articular cartilage, such as for example articular cartilage of a knee or elbow.

The size of a cartilage injury or cartilage defect may be at least 1 cm², or at least 2 cm², or at least 3 cm², or at least 4 cm², or at least 5 cm², or at least 6 cm² for example in the case of an adult human. In addition to these minimum values, the size of a cartilage injury or cartilage defect may be not more than 10 cm², or not more than 9 cm², or not more than 8 m², or not more than 7 cm², or not more than 6 cm², or not more than 5 cm² for example. In certain embodiments, the size of a cartilage injury or cartilage defect in an adult human may be at least 4 cm².

A cartilage injury or cartilage defect may be damage not only to cartilage but also to subchondral bone. In this case, the composition may be used to treat injury to subchondral bone.

“Cartilage injury treatment” means promoting repair and/or regeneration of damaged cartilage tissue and relieving or curing a state or clinical symptoms of disease or injury. Unless otherwise specified, “treatment” includes improving, alleviating, reducing, suppressing the progress of, preventing and/or suppressing the recurrence of cartilage injury or cartilage-related disease. That is, the composition of the invention can be used to improve, alleviate, reduce, suppress the progress of, prevent and/or suppress the recurrence of cartilage-related disease.

The composition of the invention can also be used to repair cartilage tissue and/or subchondral bone, regenerate cartilage tissue and/or subchondral bone, suppress the progress of degeneration in cartilage tissue and/or subchondral bone, regenerate hyaline cartilage, alleviate pain, alleviate postoperative pain, reduce dysfunction, alleviate clinical symptoms and the like.

“Repairing cartilage tissue (injury)” and “regenerating cartilage tissue (injury)” mean causing damaged or defective cartilage tissue to resemble more normal cartilage tissue. However, it is not absolutely necessary to restore absolutely normal cartilage tissue, and it is sufficient that the composition, shape, function and the like of the cartilage tissue resemble normal cartilage tissue in comparison to the damaged state before application of the composition.

“Regenerating hyaline cartilage” in the present invention is aimed at regenerating cartilage having high ratio of hyaline cartilage in comparison with fibrocartilage, with the aim of regenerating cartilage tissue rich in type II collagen and proteoglycans. In one of the preferred embodiments, the composition of cartilage that has been regenerated by application of the composition is preferably similar to the composition of normal, natural cartilage. The composition may also be a “cartilage repairing composition” or “hyaline cartilage regenerating composition” or the like for example.

A “cartilage-related disease” is a disease or state associated with cartilage injury and/or injury to subchondral bone, without any particular limitations, and examples include osteoarthritis, traumatic cartilage defect, peripheral traumatic cartilage defect, traumatic cartilage injury, osteonecrosis, osteochondritis dissecans, subchondral bone defect, degeneration of cartilage and/or subchondral bone, and misalignment in the joints.

“In combination with a concentrated bone marrow aspirate” means combined use, and may mean either that the monovalent metal salt of alginic acid (hereunder sometimes called the “component (a)”) and the concentrated bone marrow aspirate (hereunder sometimes called the “component (b)”) are contained together when the composition containing the monovalent metal salt of alginic acid is applied to a cartilage injury lesion, or that both the component (a) and the component (b) are present on the cartilage injury lesion after the composition is applied to the cartilage injury lesion, without any particular limitations. For example, (1) the component (a) and component (b) may be mixed together and then applied to an injury site, or (2) the component (a) and component (b) may each be applied to the injury site without being mixed together. In this case, for example (2-1) the component (a) and component (b) may be applied at the same time, or (2-2) the component (a) may be applied after the component (b), or (2-3) the component (b) may be applied after the component (a), or (2-4) at least one of the component (a) and component (b) may be applied multiple times.

“In combination with a concentrated bone marrow aspirate” does not specifically refer to the form at the distribution stage, including sales. For example, a composition containing the component (a) may be sold commercially, while bone marrow aspirate may be collected from a patient and made into the concentrated bone marrow aspirate of the component (b) for use.

A “subject” is a human or a non-human organism such as a bird or non-human mammal (for example, a cow, monkey, cat, mouse, rat, guinea pig, hamster, pig, dog, rabbit, sheep or horse).

To “apply the composition to a cartilage injury lesion” means to bring the composition into contact with a damaged cartilage site, and preferably means to pack the damaged cartilage site with a sufficient amount to fill up the damaged cartilage site (or defect).

To “apply a concentrated bone marrow aspirate to a cartilage injury lesion” means to bring the concentrated bone marrow aspirate into contact with the damaged cartilage site, or to bring the concentrated bone marrow aspirate into contact with the composition when the composition is in contact with the damaged cartilage site.

To “bring a curing agent into contact with at least part of the composition” means to bring a curing agent into contact with at least part of the surface of the composition after the composition has been applied to a damaged cartilage site.

“Containing a monovalent metal salt of alginic acid” means that the composition contains an amount of a monovalent metal salt of alginic acid that is effective for treating cartilage injury at a damaged cartilage site to which the composition has been applied in combination with a concentrated bone marrow aspirate.

The composition may be provided in the form of a solution using a solvent or may be provided in a dried form such as a lyophilized form (particularly a lyophilized powder). When it is provided in a dried form, a solvent is used at the time of application to make the composition into a flowable state such as a solution. The solvent is not particularly limited as long as it is applicable to a living body, but examples include injectable water, purified water, distilled water, ion-exchange water (or deionized water), milli-Q water, physiological saline, phosphate-buffered saline (PBS) and the like. Injectable water, distilled water, physiological saline and the like are preferred because they can be used in treating humans and other animals.

Using the composition of certain embodiments, the cartilage injury treatment effects may be better than those achieved using only a monovalent metal salt of alginic acid even when the cartilage injury is relatively large (such as at least 4 cm² in the case of an adult human). Moreover, by using the composition of certain embodiments it is possible to treat both cartilage and subchondral bone when the cartilage injury includes injury to the subchondral bone. The composition is easy to apply to a cartilage injury lesion since it has favorable adhesiveness to cartilage injury lesions and can also be applied with a syringe or the like. When the cartilage injury lesion is articular cartilage, the composition can be administered under arthroscopy, and extensive incision of the affected area can be avoided.

2. Monovalent Metal Salt of Alginic Acid

A “monovalent metal salt of alginic acid” is a water-soluble salt produced by ion-exchanging the hydrogen atoms of the 6-position carboxylic acid of alginic acid with a monovalent metal ion such as Na⁺ or K⁺. Specific examples of monovalent metal salts of alginic acid include sodium alginate, potassium alginate and the like, and commercially available sodium alginate is particularly desirable. A solution of the monovalent metal salt of alginic acid forms a gel when mixed with a curing agent.

“Alginic acid” is a biodegradable, high molecular weight polysaccharide that is a polymer obtained by linearly polymerizing two types of uronic acids in the form of D-mannuronic acid (M) and L-gluronic acid (G). “Alginic acid” in this Description may include alginate salts such as monovalent metal salts of alginic acid. More specifically, the alginic acid is a block copolymer in which a homopolymer fraction of D-mannuronic acid (MM fraction), homopolymer fraction of L-gluronic acid (GG fraction) and fraction in which D-mannuronic acid and L-gluronic acid are randomly arranged (MG fraction) are linked arbitrarily. The composite ratio of the D-mannuronic acid to the L-gluronic acid of the alginic acid (M/G ratio) mainly varies according to the type of algae or other organism serving as the origin thereof, is affected by the habitat and season of that organism, and extends over a wide range from a high G type having an M/G ratio of about 0.4 to a high M type having an M/G ratio of about 5.

Although the alginic acid may be of a natural origin or synthetic, it is preferably derived from a natural origin. Examples of naturally-occurring alginic acids include those extracted from brown algae. Although brown algae containing alginic acid are prominently found along seacoasts throughout the world, algae that can actually be used as raw materials of alginic acid are limited, with typical examples thereof including Lessonia found in South America, Macrocystis found in North America, Laminaria and Ascophyllum found in Europe, and Durvillea found in Australia. Examples of brown algae serving as raw materials of alginic acid include genus Lessonia, genus Macrocystis, genus Laminaria, genus Ascophyllum, genus Durvillea, genus Eisenia and genus Ecklonia.

Monovalent metal salts of alginic acid are high-molecular-weight polysaccharides, and their molecular weights are hard to determine accurately, but an appropriate molecular weight is desirable because if the molecular weight is too low the viscosity declines, potentially weakening adhesiveness to the surrounding tissue at the target site, while if the molecular weight is too high manufacturing becomes difficult, solubility declines, the handling properties are worse because the viscosity is too high in solution, and the physical properties are hard to maintain during long-term storage among other problems.

It is known that in molecular weight measurement of naturally derived high molecular weight substances, values may differ depending on the measurement method. For example, based on the effects shown in the examples, the weight-average molecular weight as measured by gel permeation chromatography (GPC) or gel filtration chromatography (which together are sometimes called size exclusion chromatography) is preferably at least 100,000, or more preferably at least 500,000, and is preferably not more than 5,000,000, or more preferably not more than 3,000,000. The preferred range is from 100,000 to 5,000,000, or more preferably from 500,000 to 3,500,000. In numerical ranges indicated by “from x to y” in this Description, the numbers represented by x and y are the minimum and maximum values.

The absolute weight-average molecular weight can also be measured by GPC-MALS, a method which combines gel permeation chromatography (GPC) and Multi Angle Light Scattering (MALS). Based on the results shown in the examples, the weight-average molecular weight (absolute molecular weight) as measured by GPC-MALS is preferably at least 10,000, or more preferably at least 80,000, or still more preferably at least 90,000, and is preferably not more than 1,000,000, or more preferably not more than 800,000, or still more preferably not more than 700,000, or yet more preferably not more than 500,000. The preferred range is from 10,000 to 1,000,000, or more preferably from 80,000 to 800,000, or still more preferably from 90,000 to 700,000, or yet more preferably from 90,000 to 500,000.

When calculating the molecular weight of a high-molecular-weight polysaccharide by such methods, a measurement error of at least 10 to 20% is normal. For example, a value of 400,000 may vary in the range of 320,000 to 480,000, a value of 500,000 may vary in the range of 400,000 to 600,000, and a value of 1,000,000 may vary in the range of 800,000 to 1,200,000 for example.

The molecular weights of monovalent metal salts of alginic acid can be measured by ordinary methods.

Typical conditions for measuring molecular weight by gel permeation chromatography are described in the examples of the Description. For example, GMPW-XL×2+G2500PW-XL (7.8 mm I.D.×300 mm) may be used as the columns, a 200 mM sodium nitrate aqueous solution as the eluent, and pullulan as the molecular weight standard.

Typical conditions for measuring molecular weight by GPC-MALS are described in the examples of the Description. An RI detector and a light scattering detector (MALS) may be used as the detectors.

The apparent viscosity of the monovalent metal salt of alginic acid is preferably an apparent viscosity of 40 mPa·s to 800 mPa·s, or more preferably 50 mPa·s to 600 mPa·s as measured with a cone plate viscometer at 20° C. in a 1 w/w % solution of the monovalent metal salt of alginic acid dissolved in Milli-Q water. The conditions described below may be used as the conditions for measuring apparent viscosity. In this Description, “apparent viscosity” may be called simply “viscosity”.

3. Endotoxin Reduction Treatment

In certain embodiments, the monovalent metal salt of alginic acid is a low endotoxin monovalent metal salt of alginic acid. Low endotoxin refers to that in which the endotoxin level thereof has been substantially lowered to an extent that does not induce inflammation or fever. More preferably, the monovalent metal salt of an alginic acid is preferably subjected to an endotoxin reduction treatment.

Endotoxin reduction treatment can be carried out by a known method or a method complying therewith. For example, this treatment can be carried out by the method of Suga et al. involving purification of sodium hyaluronate (see, for example, Japanese Patent Application Laid-open No. H9-324001), the method of Yoshida et al. involving purification of β1,3-glucan (see, for example, Japanese Patent Application Laid-open No. H8-269102), the method of William et al. involving purification of a biopolymer such as alginate or gellan gum (see, for example, Published Japanese Translation No. 2002-530440 of PCT International Publication), the method of James et al. involving purification of polysaccharide (see, for example, International Publication No. 93/13136 pamphlet), the method of Lewis et al. (see, for example, U.S. Pat. No. 5,589,591), the method of Hermanfranck et al. involving purification of alginate (see, for example, Appl. Microbiol. Biotechnol. (1994), 40:638-643) or a method complying therewith. The endotoxin reduction treatment of the present invention is not limited thereto, but rather can be carried out by a known method such as cleaning, purification using filtration with filter (endotoxin removing filter or electrification filter), ultrafiltration or a column (such as an endotoxin adsorption affinity column, gel filtration column or ion exchange column), adsorption to a hydrophobic substance, resin or activated carbon and the like, organic solvent treatment (such as extraction with an organic solvent or precipitation or deposition by addition of organic solvent), surfactant treatment (see, for example, Japanese Patent Application Laid-open No. 2005-036036) or a suitable combination thereof. A known method such as centrifugal separation may be suitably combined with these treatment steps. Endotoxin reduction treatment is preferably suitably selected according to the type of alginic acid.

The endotoxin level can be confirmed by a known method, and can be measured using a known method such as a method using Limulus reagent (LAL) or Endospecy (registered trademark) ES-24S set (Seikagaku Corporation).

Although there are no particular limitations on the endotoxin treatment method of the alginic acid, the endotoxin content of the monovalent metal salt of alginic acid in the case of measuring endotoxin using a limulus reagent (LAL) is preferably 500 endotoxin units (EU)/g or less, more preferably 100 EU/g or less, even more preferably 50 EU/g or less and particularly preferably 30 EU/g or less as a result thereof. Sodium alginate that has undergone endotoxin reduction treatment can be acquired as a commercially available products such as Sea Matrix (registered trademark) (Mochida Pharmaceutical), PRONOVA™ UP LVG (FMC BioPolymer) or the like.

4. Preparation of Solution of Monovalent Metal Salt of Alginic Acid

The composition may be prepared by using a solution of a monovalent metal salt of alginic acid. The solution of a monovalent metal salt of alginic acid can be prepared by a known method or method complying therewith. Namely, the monovalent metal salt of alginic acid used in the present invention can be produced by a known method such as an acid method or calcium method using the previously described brown algae. More specifically, after extracting from these brown algae using an alkaline aqueous solution such as aqueous sodium carbonate solution, for example, alginic acid be obtained by adding an acid (such as hydrochloric acid or sulfuric acid), and a salt of alginic acid can be obtained by ion exchange of the alginic acid. Endotoxin reduction treatment is then carried out as previously described. There are no particular limitations on the solvent of the monovalent metal salt of alginic acid provided it is a solvent that can be applied in vivo, and examples of such solvents include purified water, distilled water, ion exchange water, Milli-Q water, physiological saline and phosphate-buffered saline (PBS). These are preferably sterilized and preferably subjected to endotoxin reduction treatment. For example, Milli-Q water can be used after sterilizing by filtration.

When the composition is provided in a dry state as a lyophilizate or the like, the above-described solvent can be used to prepare it into a solution having fluidity.

Moreover, all of the operations for obtaining the composition are preferably carried out in an environment at a low endotoxin level and a low bacterial level. For example, the operations are preferably carried out in a clean bench using sterilized tools. The tools used may be treated with a commercially available endotoxin removal agent.

5. Apparent Viscosity of Composition

In certain embodiments, the composition is in a liquid form having fluidity, or in other words a solution, when it is applied to a cartilage injury lesion. When the composition is first mixed with a concentrated bone marrow aspirate before being applied to a cartilage injury lesion, the mixture preferably has fluidity. To ensure accurate viscosity measurement, when the composition is mixed with a material such as concentrated bone marrow aspirate or cells that does not dissolve in a solvent and then applied to a damaged part, the apparent viscosity of the composition is preferably given as the viscosity of the composition containing the monovalent metal salt of alginic acid before it is mixed with the concentrated bone marrow aspirate or the like. When the composition and the concentrated bone marrow aspirate are each applied to a damaged part without being mixed before administration, the apparent viscosity of the composition is the viscosity of the composition containing the monovalent metal salt of alginic acid. This preferred apparent viscosity of the composition is not particularly limited as long as it is appropriate for a cartilage injury lesion but considering adhesiveness with the surrounding tissue of the target site, it is preferably at least 10 mPa·s, or more preferably at least 100 mPa·s, or still more preferably at least 200 mPa·s, or especially at least 500 mPa·s as measured at 20° C. under the conditions described below using a rotational viscometer (cone plate viscometer). Considering the handling properties, the apparent viscosity of the composition is preferably not more than 100,000 mPa·s, or still more preferably not more than 50,000 mPa·s, or yet more preferably not more than 20,000 mPa·s, or even more preferably not more than 10,000 mPa·s, or especially not more than 7,000 Pa·s. Application with a syringe or the like is easier if the apparent viscosity is not more than 20,000 mPa·s. However, even if the apparent viscosity is 20,000 mPa·s or more, the composition can still be applied using an appropriate means such as a pressure mold or electric filling device. The preferred range of the composition is from 10 mPa·s to 50,000 mPa·s, or more preferably from 100 mPa·s to 30,000 mPa·s, or still more preferably from 200 mPa·s to 20,000 mPa·s, or yet more preferably from 500 mPa·s to 20,000 mPa·s, or especially from 700 mPa·s to 20,000 mPa·s. In another preferred embodiment, it may be from 500 mPa·s to 10,000 mPa·s, or from 2,000 mPa·s to 10,000 mPa·s. Certain embodiments of the composition have a viscosity that allows application to a subject with a syringe or the like.

It is particularly desirable to measure the apparent viscosity of the composition with a cone plate viscometer using a 35/1 sensor (cone diameter 35 mm,)1°. Preferably this is performed in accordance with the viscosity measurement methods of the Japanese Pharmacopoeia (16th Edition). For example, measurement is preferably performed under the following measurement conditions. When a solvent is necessary for viscosity measurement, the sample solution is preferably prepared using Milli-Q water. The measurement temperature is 20° C. The rotational speed of the cone plate viscometer is determined based on a benchmark rate of 1 rpm when measuring a 1% solution of the monovalent metal salt of alginic acid and 0.5 rpm when measuring a 2% solution. For the reading time, measurement is performed for 2 minutes when measuring a 1% solution of the monovalent metal salt of alginic acid, and the average of the values is taken during the period from 1 to 2 minutes after the start of measurement. When measuring a 2% solution, measurement is performed for 2.5 minutes, and the average of the values is taken during the period from 0.5 to 2.5 minutes after the start of measurement. Each test value is the average of three times of measurements. When the viscosity of the composition is 10,000 mPa·s or more, measurement is preferably performed for 2.5 minutes at 0.5 rpm with a 20/1 sensor (cone diameter 20 mm,)1°, and the averages of the values is taken from 0.5 to 2.5 minutes after the start of measurement.

Because the apparent viscosity of a solution of a monovalent metal salt of alginic acid is affected by the M/G ratio, an alginic acid having the desired M/G ratio can be selected according to the solution viscosity and the like for example. The M/G ratio of the alginic acid used in the present invention is about 0.1 to 5.0, or preferably about 0.1 to 4.0, or more preferably about 0.2 to 3.5.

As described above, since the M/G ratio is mainly determined by the species of the seaweed, the species of the brown alga used as the raw material affects the viscosity of the monovalent metal salt solution of alginic acid. The alginic acid used with the present invention is preferably derived from a brown alga of genus Lessonia, genus Macrycystis, genus Laminaria, genus Ascophyllum and genus Durvillea, more preferably from a brown alga of genus Lessonia, and particularly preferably derived from Lessonia nigrescens.

6. Concentrated Bone Marrow Aspirate (cBMA)

The composition is used in combination with a concentrated bone marrow fluid (aspirate).

Concentrated bone marrow aspirate (cBMA), also called bone marrow aspirate concentrate (BMAC), is a concentrated cell aspirate containing liquid growth factors and bone marrow-derived mesenchymal stem cells (MSCs) and is obtained by centrifugation from bone marrow aspirate. cBMA can be prepared simply by centrifuging bone marrow aspirate. It therefore does not require ex vivo cell culture and can be used for first-term treatment. Moreover, it can also contain a sufficient number of cells because it is concentrated at the preparation stage. In certain embodiments, the concentrated cell aspirate is autologous concentrated cell aspirate, or in other words derived from the subject.

The method for preparing the concentrated bone marrow aspirate is not particularly limited, and for example it may be prepared as follows.

A concentrated bone marrow aspirate can be prepared according to the protocols described in Chahla J et al., Arthrosc Tech (2017) 6 (2), e441-e445. Specifically, a syringe is used to collect bone marrow aspirate from the ilium of a subject, and coagulated blood in the bone marrow fluid is removed by filtration. The filtered bone marrow fluid is centrifuged, and a cell fluid is then aspirated centered on the intermediate monocyte layer (buffy coat) and including a little of the overlying plasma component (PRP) but taking care to avoid the underlying red blood cells (RBC). This is then used as the concentrated bone marrow aspirate.

This can also be prepared using a commercial machine for preparing concentrated bone marrow aspirate from bone marrow aspirate, such as the Bio-Cue System (Biomet Biologics, Mrtin J R et al., 2013; 54:219-24) or CenTrate (R) BMA Device.

The number of bone marrow mesenchymal stem cells in the concentrated bone marrow aspirate is preferably from 1×10¹ to 1×10⁹ cells/mL, or more preferably from 1×10² to 1×10⁷ cells/mL, or still more preferably from 1×10³ to 1×10⁶ cells/mL. In certain embodiments, the number of bone marrow mesenchymal stem cells in the concentrated bone marrow aspirate is from 1×10³ to 1×10⁵ cells/mL.

7. Preparation of Composition

The composition contains a monovalent metal salt of alginic acid. The inventors first discovered that when a monovalent metal salt of alginic acid is administered to a cartilage injury lesion in vivo in combination with a concentrated bone marrow aspirate, it has the effect of regenerating or treating the cartilage tissue even when the cartilage injury lesion is relatively large. Preferably the monovalent metal salt of alginic acid may be contained in the composition in an amount sufficient to have a regenerative or therapeutic effect on cartilage tissue when administered to an affected area in combination with a concentrated bone marrow aspirate, and preferably the monovalent metal salt of alginic acid constitutes at least 0.1 w/v %, or more preferably at least 0.5 w/v %, or still more preferably at least 1 w/v % of the composition as a whole before it is combined with the concentrated bone marrow aspirate. The preferred concentration of the monovalent metal salt of alginic acid in the composition before it is combined with the concentrated bone marrow aspirate cannot be set unconditionally because it depends partly on the molecular weight, but is preferably from 0.5 w/v % to 5 w/v %, or more preferably from 1 w/v % to 5 w/v %, or still more preferably from 1 w/v % to 4 w/v %, or especially from 1 w/v % to 3 w/v %. In a different embodiment, the concentration of the monovalent metal salt of alginic acid in the composition may preferably be from 0.5 w/w % to 5 w/w %, or more preferably from 1 w/w % to 5 w/w %, or still more preferably from 1 w/w % to 4 w/w %, or especially from 1 w/w % to 3 w/w %. When the composition is mixed with a concentrated bone marrow aspirate and then administered to a cartilage injury lesion, the mixture is preferably prepared so that the concentration of the monovalent metal salt of alginic acid is within the above range.

When a composition is prepared as described above using a monovalent metal salt of alginic acid that has been purified until it exhibits the desired endotoxin level, the endotoxin content of the composition is normally not more than 500 EU/g, or preferably not more than 300 EU/g, or still more preferably not more than 150 EU/g, or especially not more than 100 EU/g.

In one embodiment, the composition does not contain any components other than the monovalent metal salt of alginic acid that have a pharmacological effect on damaged cartilage tissue except when the composition is used in combination with a concentrated bone marrow aspirate. Even in this case, the composition still has a sufficient regenerative or therapeutic effect on cartilage injury lesions.

In certain embodiments, other pharmacologically active components and components normally used in drugs, such as conventional stabilizers, emulsifiers, osmotic pressure adjusters, buffers, isotonic agents, preservatives, pain relievers and colorants, may be included in the composition as appropriate.

8. Application of Composition

Preferably the damaged cartilage area is made visible by a means such as incision, arthroscopy or endoscopy before the composition is applied to the cartilage injury lesion. An arthroscope is a kind of endoscope for observing the condition of a joint.

A step of removing unnecessary tissue from the cartilage injury lesion and its margins may be included as necessary before application of the composition to the cartilage injury lesion. “Unnecessary tissue from the cartilage injury lesion and its margins” here means lesion tissue of the damaged cartilage site, parts where the margins have been repaired with fibrocartilage, and parts where the cartilage is unstable.

The affected area may also be washed as necessary before applying the composition. “Washing the affected area” means using physiological saline or the like to remove blood components and other unnecessary tissue and the like from the site where the composition is to be applied. After being washed, the affected area is preferably dried by wiping off unnecessary residual liquid components and the like before applying the composition.

The cartilage injury lesion may also be subjected to bone marrow stimulation as necessary before the composition is applied to the cartilage injury lesion. Bone marrow stimulation technique is a therapeutic method whereby one or more defects extending into subchondral bone (full-thickness defects) are created by picking, drilling or the like of the damaged site, and is intended to stimulate bleeding from the bone marrow and migration of cartilage progenitor cells from the bone marrow to the cartilage injury lesion.

In this Description, “applying the composition to the cartilage injury lesion” means bringing the composition into contact with the cartilage injury lesion, and preferably means filling the cartilage injury lesion with a sufficient quantity of the composition to cover the cartilage injury lesion (or defect). Alternatively, one or more relatively small holes may be formed in the cartilage injury lesion (or defect), and the composition may be poured into the holes so as to cover the holes. When applied to the cartilage injury lesion, the composition is preferably poured into so as to thoroughly fill the entire cavity volume of the affected area. Furthermore, the liquid surface of the composition is preferably implanted in the cartilage injury lesion up to about the same height as the surrounding cartilage tissue. “Applying” in this description is used in a sense that includes the sense of “implanting”.

When applied to the cartilage injury lesion, the composition is preferably in a liquid form having fluidity, or in other words in the form of a solution. “Having fluidity” means having the property of changing into an amorphous form, and does not necessarily mean having the property of constantly flowing. For example, preferably the composition has fluidity that allows it to be enclosed in a syringe or the like and injected into the cartilage injury lesion. When the composition is mixed with a concentrated bone marrow aspirate and applied to the cartilage injury lesion, preferably the mixture has fluidity.

In one of the certain embodiments, the composition has fluidity that allows it to be left for 1 hour at 20° C. and then injected into the cartilage injury lesion with a syringe equipped with a 14G to 26G injection needle, and more preferably with a 21G injection needle. When the composition is provided in a dried form such as a lyophilized form, a solvent or the like may be used at the time of application to obtain a composition with fluidity as discussed above. Because the composition of certain embodiments is in the form of a solution, it can be fitted to a cartilage injury lesion of any shape and can also fill the entire cartilage injury lesion.

The composition in solution form can be applied easily to the cartilage injury lesion with a syringe, gel pipette, dedicated syringe, dedicated injection device, filling device or the like.

When the composition is highly viscous and difficult to apply with a syringe, a pressurized type or electric type syringe or the like may be used. It may also be applied to the cartilage injury lesion with a stick or the like rather than a syringe for example. When injecting with a syringe, it is desirable to use a 14G to 27G needle or a 14G to 26G needle.

The amount of the composition that is applied is not particularly limited and can be determined according to the volume of the application site of the subject's cartilage injury lesion, but for example the amount may be from 0.1 mL to 10 mL, or preferably from 0.2 mL to 8 mL, or more preferably from 0.2 mL to 5 mL for example. When the composition is applied to the cartilage injury lesion, it is preferably injected so as to thoroughly fill the defect volume of the cartilage injury lesion.

The amount of the concentrated bone marrow aspirate that is applied is not particularly limited and can be determined according to the volume of the application site of the subject's cartilage injury lesion, but is preferably from 0.1 mL to 10 mL, or more preferably from 0.2 mL to 8 mL, or still more preferably from 0.2 mL to 5 mL for example. When the concentrated bone marrow aspirate and the composition are administered separately without being mixed, the concentrated bone marrow aspirate can be applied to the cartilage injury lesion with a device such as a syringe.

When the two are combined, the ratio of the applied amounts of the composition and the concentrated bone marrow aspirate is not particularly limited, but for example the ratio of the composition (mL) to the concentrated bone marrow aspirate (mL) is from 1:5 to 10:1, or preferably from 1:2 to 5:1.

In another embodiment, the applied amount of the composition is preferably 20% to 80% or more preferably from 30% to 70% of the total amount (mL) of the combined amount of the composition and the concentrated bone marrow aspirate.

In another embodiment, the concentration of the monovalent metal salt of alginic acid relative to the total amount (mL) of the applied composition and the concentrated bone marrow aspirate is preferably from 0.5 w/w % to 5 w/w %, or more preferably from 1 w/w % to 5 w/w %, or still more preferably from 1 w/w % to 4 w/w %, or especially from 1 w/w % to 3 w/w %.

The “total amount” here may be either the total amount of a mixture of the composition and the concentrated bone marrow aspirate or the total amount of the two when they are applied separately.

In another embodiment, when the monovalent metal salt of alginic acid is in a lyophilized form, the above ratio or concentration is preferably adjusted by using the concentrated bone marrow aspirate and a solvent such as injectable water to dissolve the lyophilized form.

After being applied to the cartilage injury lesion, the composition is used by bringing a curing agent into contact with at least part of the composition having fluidity. This step serves to gel and cure the surface of the composition containing the monovalent metal salt of alginic acid, thereby preventing leakage or detachment of the composition from the affected part. The use of the curing agent is not limited as long as this effect is obtained, but preferably the curing agent is brought into contact with the entire surface of the composition. Moreover, preferably the entire surface of the composition is cured immediately after contact of the curing agent with the composition surface, leaving the rest of the composition in a sol form.

Preferably the composition does not contain an amount of the curing agent that cures the composition before it is applied to the cartilage injury lesion of a subject. Thus, the composition may contain an amount of the curing agent that does not cure the composition even after passage of a certain amount of time. The certain amount of time here is not particularly limited but is preferably about 30 minutes to 12 hours. Not containing an amount of a curing agent that cures the composition may mean for example that the composition can be injected with a syringe equipped with a 21G needle after having been left for 1 hour at 20° C. Certain embodiments of the composition do not contain a curing agent.

The curing agent is not particularly limited as long as it can solidify the surface of a solution of the monovalent metal salt of alginic acid by crosslinking. Examples of curing agents include compounds of divalent and higher metal ions such as Ca²⁺, Mg²⁺, Ba²⁺ and Sr²⁺, and crosslinkable reagents having 2 to 4 amino groups in the molecule. More specific examples of divalent and higher metal ion compounds include CaCl₂, MgCl₂, CaSO₄ and BaCl₂, while crosslinkable reagents having 2 to 4 amino groups in the molecule also include dialkanes that may also have a lysyl group (—COCH(NH₂)-(CH₂)₄-NH₂) on a nitrogen atom, or in other words diaminoalkanes and diaminoalkane derivatives in which an amino group is substituted with a lysyl group to form a lysylamino group, with specific examples including diaminoethane, diaminopropane, N-(lysyl)-diaminoethane and the like, but a CaCl₂ solution is especially desirable for reasons of availability and strong gel formation.

In one of the certain embodiments, the curing agent is preferably brought into contact with the surface of the composition after the composition has been applied to the cartilage injury lesion. The method of bringing the curing agent (such as a divalent or higher metal ion) into contact with part of the composition is not particularly limited, but for example a solution of a divalent or higher metal ion may be applied to the surface of the composition with a syringe, sprayer or the like. For example, the curing agent may be applied gradually and continuously to the surface of the composition for a few seconds to several 10 seconds. This may be followed as necessary by a treatment to remove residual curing agent from near the cartilage injury lesion. The curing agent may be removed for example by washing the application site with physiological saline or the like.

The amount of the curing agent used is preferably adjusted appropriately according to the size, shape, location and the like of the cartilage injury lesion. The amount of the curing agent is adjusted so as not to be excessive so that the curing agent does not strongly affect the tissue surrounding the cartilage injury lesion. The amount of the divalent or higher metal ion used is not particularly limited as long as it is sufficient to cure the composition containing the monovalent metal salt of alginic acid. However, when using a 100 mM CaCl₂ solution for example, the amount of CaCl₂ solution used is preferably about 0.3 mL to 10 mL or more preferably about 1 mL to 5 mL when the cartilage injury lesion is about 4 cm² in size. The amount may be suitable increased and decreased while observing the state of the composition at the application site.

When the curing agent contains calcium, it is known that gelling is more rapid and a harder gel is formed the greater the calcium concentration. Because calcium has cytotoxicity, however, there is a risk that the cartilage regeneration effects of the composition may be adversely affected if the concentration is too high. Therefore, when using a CaCl₂ solution for example to cure the surface of a composition containing a monovalent metal salt of alginic acid, a concentration of 25 mM to 200 mM or more preferably 50 mM to 150 mM is desirable.

After the curing agent is added to the composition, it is preferably left standing for a certain amount of time, after which residual curing agent at the addition site is removed by washing or the like. The standing time is not particularly limited, but preferably the surface of the composition is gelled by being left standing for at least about 1 minute, or more preferably at least about 4 minutes. A standing time of about 1 minute to 10 minutes, or preferably about 4 to 10 minutes, or 4 to 7 minutes, or still more preferably about 5 minutes is also desirable. During this certain amount of time the composition and the curing agent are preferably in contact with each other, and additional curing agent may also be added so that the liquid surface of the composition does not dry out.

Because the composition is in solution form, it can be easily applied to a site of any shape, and the entire application site may be covered with the composition, which also has favorable adhesiveness with surrounding tissue. The calcium concentration of the part of the composition that contacts the surrounding tissue may be kept low, so there is little problem with calcium cytotoxicity. Because the part of the composition that contacts the surrounding tissue is little affected by the curing agent, the composition can easily contact the cells and tissue of the application site, thereby enhancing the therapeutic effects of the composition. Preferably the composition is highly biocompatible and fuses with living tissue to a degree that it cannot be distinguished at the application site about 4 weeks after it is applied to a cartilage injury lesion.

The affected site may also be sutured as necessary.

The number and frequency of applications of the composition may be increased and decreased depending on the symptoms and effects. For example, it may be applied only once, or it may be applied intermittently once a month to once a year.

Because alginate is a substance that is not originally present in the bodies of animals, animals do not carry enzymes that specifically degrade alginic acid. Although alginate is gradually degraded by ordinary hydrolysis in the bodies of animals, it is degraded in the body more slowly than polymers such as hyaluronic acid, and because cartilage does not contain blood vessels, long-term continuous effects can be expected when a cartilage injury lesion is filled with alginate.

In certain embodiments, cells, growth factors and the like may be used in combination with the composition in addition to those contained in the concentrated bone marrow aspirate when the composition is applied to the cartilage injury lesion, and cell death inhibitors and the other drugs described below and the like may also be used in combination with the composition. In another aspect of the invention, however, embodiments that do not use the composition in combination with such cells and factors are also desirable.

The cells are not particularly limited as long as they are useful for treating cartilage injury, and examples include cartilage cells, cartilage precursor cells, stem cells, stromal cells, mesenchymal stem cells, bone marrow stromal cells, synovial cells, ES cells, iPS cells and the like. The origin is not particular limited, and they may be derived from bone marrow, fatty tissue, umbilical cord blood or the like. Bone marrow mesenchymal stem cells and/or bone marrow stromal cells are more preferred.

In addition to those contained in the concentrated bone marrow aspirate, factors that promote cell growth may also be included in the composition. Examples of such factors include BMP, FGF, VEGF, HGF, TGF-β, IGF-1, PDGF, CDMP (cartilage-derived morphogenetic protein), CSF, EPO, IL, PRP (platelet rich plasma), SOX, IF and the like.

The cartilage injury lesion may also be filled with drugs including antibiotics such as streptomycin, penicillin, tobramycin, amikacin, gentamicin, neomycin and amphotericin B, anti-inflammatories such as aspirin, non-steroidal anti-inflammatory drugs (NSAIDs) and acetaminophen, and proteolytic enzymes, corticosteroids and HMG-CoA reductase inhibitors such as simvastatin and lovastatin and the like either before, during or after application of the composition. These drugs may also be used in a mixture with the composition. They may also be administered in combination orally or parenterally. In addition, muscle relaxants, opioid pain relievers, neuropathic pain relievers and the like may also be administered in combination either orally or parenterally as necessary.

These preferred embodiments of the composition and methods for using the composition and the like conform to the descriptions above.

9. Treatment Method

A method for using the composition to treat cartilage injury is also provided here. Preferably this is a method for treating cartilage injury in a subject by applying a composition containing a monovalent metal salt of alginic acid to a cartilage injury lesion of the subject in combination with a concentrated bone marrow aspirate, wherein the composition has fluidity when applied to the cartilage injury lesion, and more preferably the composition is used by bringing a curing agent into contact with at least part of the surface of the composition after it is applied to the cartilage injury lesion.

In certain embodiments, the following steps are preferably included.
(a) A step of making the cartilage injury lesion or cartilage defect visible by incision, arthroscopy or endoscopy;
(b) A step of removing unnecessary tissue from the cartilage injury lesion and its margins as necessary;
(c) A step of applying a concentrated bone marrow aspirate in combination with a composition containing a monovalent metal salt of alginic acid;
(d) A step of bringing a curing agent into contact with the surface of the implanted composition;
(e) A step of washing the site where the curing agent has contacted the composition; and
(g) A step of suturing as necessary.

Preferred embodiments of the composition, specific methods of application to the cartilage injury lesion, methods for curing the compositions, definitions of terms and the like are as given above. This treatment method may also be implemented appropriately in combination with other methods and drugs for treating cartilage injury.

In certain embodiments, the cells, factors that promote cell growth, and other drugs and the like described above may be applied to the cartilage injury lesion together with the composition. In certain other aspects of the invention, however, embodiments that do not use such additional cells, additional factors and other drugs in combination with the composition are also desirable. Preferred embodiments of the composition can promote cartilage regeneration even without the use of such additional cells, additional factors and other drugs.

The use of a monovalent metal salt of alginic acid to manufacture the composition is also provided.

This use is a use of a monovalent metal salt of alginic acid to manufacture a composition for treating cartilage injury, wherein the composition is applied to a cartilage injury lesion of a subject in combination with a concentrated bone marrow aspirate and has fluidity when applied to the cartilage injury lesion.

Also provided here is a monovalent metal salt of alginic acid used for treating cartilage injury, wherein a composition containing the monovalent metal salt of alginic acid and having fluidity is applied together with a concentrated bone marrow aspirate to a cartilage injury lesion of a subject in need of treatment for cartilage injury.

Also provided here is a combination containing a concentrated bone marrow aspirate and also containing a composition that has fluidity and contains a monovalent metal salt of alginic acid, wherein the combination is used to treat cartilage injury by applying it to a cartilage injury lesion of a subject requiring treatment for cartilage injury.

10. Kit

The present invention provides a cartilage injury treatment kit.

The kit of the invention may contain the composition of the invention. The composition of the invention contained in the kit of the invention is preferably in a solution state or dried state. The dried state is preferably a lyophilized state, or more preferably a lyophilized powder.

When the composition of the invention is in a dried state, a solvent (such as injectable water) for dissolving the composition, a dissolving syringe and an injection needle are preferably included in the kit.

The kit of the invention may also contain a curing agent (such as a calcium chloride solution), and preferably contains a syringe and needle for the curing agent.

The kit of the invention may also contain an operating manual and the like.

A desirable example of a kit in the present invention may be a kit containing (1) a vial containing a lyophilized form of low-endotoxin sodium alginate, (2) an ampoule containing a solvent such as injectable water for dissolution, (3) an ampoule containing a divalent or higher metal ion compound such as a calcium chloride solution as a curing agent, (4) a syringe, and (5) an injection needle and the like in a single package. In another example of the kit, a monovalent metal salt of alginic acid is contained in one chamber of a syringe having two integrally molded chambers separated by a partition wall, while the other chamber contains a solvent for dissolution or a solution containing a curing agent, and the syringe is constructed so that the partition wall between the chambers can be opened easily during use so that the two sides can be mixed and dissolved as needed. In another example of the kit, a solution of the monovalent metal salt of alginic acid is contained in a pre-filled syringe and can be applied as is during use without any preparation operation. In another example of the kit, an alginate solution and a curing agent are contained in separate syringes and included together in a single pack. A vial filled with a solution of the monovalent metal salt of alginic acid may also be used. The “composition of the invention”, the “curing agent” and the like are as explained above.

All documents and publications described herein are incorporated herein by reference in their entirety, regardless of their purpose. The priority claim for this application is based on Japanese Patent Application No. 2020-005679 filed on Jan. 17, 2020, and the entire contents of the Claims, Description and drawings of that application are herein incorporated by reference.

### EXAMPLES

The invention is explained further below based on the following examples, but the invention should not be understood solely in terms of these examples.

### Example 1

### Application of Sodium Alginate Solution and Concentrated Bone Marrow Aspirate to Rabbit Osteochondral Defect Model

### 1. Methods

### 1.1. Procedures for Preparing Rabbit Osteochondral Defect Model

Using a medial parapatella approach, 3.0 cm skin incisions were made on fifty-two 24-week-old Japanese white rabbits (104 knees) under general anesthesia to expose the patellofemoral joint and the femoral trochlea. Using a dedicated sleeve under saline cooling, an osteochondral defect was created with a 5.0 mm electric drill to a depth of 2.0 mm in the center of the trochlea. This defect is a relatively large defect corresponding to a 4 cm² defect in a human.

1.2. Preparation of Sodium Alginate Solution

The following sodium alginates were used. Low-endotoxin sodium alginate with an endotoxin content of less than 50 EU/g was used. The apparent viscosity and weight-average molecular weight of each sodium alginate is shown in the table below. The apparent viscosity of the sodium alginate was measured with a rotational viscometer in accordance with the viscosity measurement methods of the Japanese Pharmacopoeia (16th Edition). The specific measurement methods are as follows. The sample solutions were prepared using Milli-Q water. A rotational cone plate viscometer (Rheostress RS600 viscosity and viscoelasticity measurement device, Thermo Haake GmbH, sensor: 35/1) was used as the measurement equipment. The rotational speed was 1 rpm when measuring a 1 w/w % sodium alginate solution and 0.5 rpm when measuring a 2 w/w % sodium alginate solution. For the reading time, measurement was performed for 2 minutes when measuring a 1 w/w % solution, and the average of the values was taken during the period from 1 to 2 minutes after the start of measurement, while when measuring a 2 w/w % solution measurement was performed for 2.5 minutes and the average of the values was taken during the period from 0.5 to 2.5 minutes after the start of measurement. The average of 3 measurements was given as the measurement value. The measurement temperature was 20° C.

The weight-average molecular weight of each sodium alginate was measured by two measurement methods, gel permeation chromatography (GPC) and GPC-MALS. The measurement conditions are as follows.

[Pre-treatment methods]

An eluent was added to dissolve the sample, which was then filtered through an 0.45 μm membrane filter to obtain a measurement solution.

(1) Gel Permeation Chromatography (GPC) Measurement

[Measurement conditions (relative molecular weight distribution measurement)]

Columns: TSKgel GMPW-XL×2+G2500PW-XL (7.8 mm I.D.×300 mm×3)

Eluent: 200 mM sodium nitrate aqueous solution

Flow rate: 1.0 mL/min

Concentration: 0.05%

Detector: RI detector

Column temperature: 40° C.

Injection volume: 200 μl

Molecular weight standard: Standard pullulan, glucose

(2) GPC-MALS Measurement

[Refractive index increment (dn/dc) measurement (measurement conditions)]

Differential refractometer: Optilab T-rEX

Measurement wavelength: 658 nm

Measurement temperature: 40° C.

Medium: 200 mM sodium nitrate aqueous solution

Sample concentration: 0.5 to 2.5 mg/mL (5 concentrations)

[Measurement conditions (absolute molecular weight distribution measurement)]

Columns: TSKgel GMPW-XL×2 +G2500PW-XL (7.8 mm I.D.×300 mm×3)

Eluent: 200 mM sodium nitrate aqueous solution

Flow rate: 1.0 mL/min

Concentration: 0.05%

Detector: RI detector, light scattering detector (MALS)

Column temperature: 40° C.

Injection volume: 200 μL

TABLE 1

| Apparent viscosity | | Weight-average | | |
|---|---|---|---|---|
| (mPa · s) | | molecular weight | | M/G |
| 1 w/w % | 2 w/w % | GPC | GPC-MALS | ratio |
| 300 to | 3000 to | 1,100,000 to | 200,000 to | 0.2 to |
| 600 | 6000 | 1,800,000 | 400,000 | 1.8 |

This low-endotoxin sodium alginate was dissolved in saline to prepare 2 w/v % solutions and 4 w/v % solutions.

1.3. Preparing mixed transplant solutions of sodium alginate mixed with cells for transplantation (mesenchymal stem cells: MSCs) or bone marrow aspirate concentrate (BMAC)

The BMAC was prepared following BMAC preparation protocols (Chahla J et al., Arthrosc Tech (2017) 6 (2), e441-e445). The rabbits were first anesthetized by intravenous administration of 0.05 mg/kg of pentobarbital, and bone marrow fluid was collected from both ilia of each rabbit with a syringe equipped with a 18G needle and containing 0.5 mL of heparin (Novo-heparin 5000 units/5 mL, Mochida Pharma. Co., Ltd., Tokyo, Japan), and passed through a strainer with a pore diameter of 200 μm to remove coagulated blood from the bone marrow fluid. 5 mL of the filtered bone marrow fluid was taken and centrifuged at 800 g for 5 minutes at 4° C., and a cell fluid was then pipetted with a pipette tip, centering on the intermediate monocyte layer (buffy coat) and including a little of the overlying plasma component (PRP) but taking care to avoid the underlying red blood cells (RBC). This was used as the BMAC.

To prepare allogeneic MSCs for transplantation, BMAC purified by the methods described above was proliferation cultured in a planar culture using MSC medium. After 2 passages, the cells were used as MSCs for transplantation (Passage 3).

The MSCs were mixed with low-endotoxin sodium alginate solution (sometimes called “UPAL”) to a final MSC concentration of 2 x 10⁴ cells/mL and a final UPAL concentration of 2 w/w % and transplanted (allogeneic transplant) into defect parts as the transplant solution for the UPAL+MSC group.

As the BMAC for transplantation, the purified BMAC cell fluid was mixed with an equal amount of 4% UPAL to a final MSC concentration of 2×10⁴ cells/mL and a final UPAL concentration of 2% and transplanted (autologous transplant) into defect parts as a transplant solution for the UPAL+BMAC group.

The MSC medium here was prepared by adding 10% FBS (10828028SP, Gibco) and antibiotics (100 U/mL penicillin G and 0.1 mg/mL streptomycin) to DMEM-High Glucose (044-29765, WAKO).

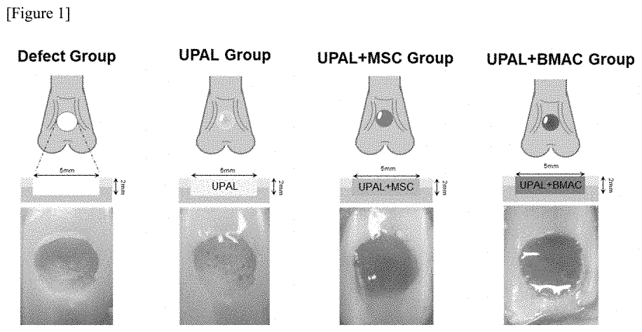

Each knee was assigned randomly to one of 4 groups, i.e., a non-treatment group (Defect group), a UPAL single transplant group (UPAL group), a UPAL and MSCs combined transplant group (UPAL+MSC group) or a UPAL and BMAC combined transplant group (UPAL+BMAC group) (FIG. 1).

In the UPAL group, UPAL+MSC group and UPAL+BMAC group, an 18G needle was used to carefully fill the defects with the transplant solution to the same height as the cartilage surrounding the transplantation site, and a 100 mM calcium chloride solution was then dripped onto the surface for 30 to 40 seconds to gel the surface of the sodium alginate solution. The surgical field was thoroughly washed with saline, and the joint capsule and skin were each sutured with 4-0 nylon thread to complete surgery. After awakening from anesthesia, the rabbits were allowed to move freely around their cages without restriction. 4 weeks and 16 weeks after surgery they were euthanized, and the knees were removed.

4 weeks after surgery, 10 knees in each group were subjected to macroscopic and histological evaluation. Micro-CT measurement was also performed to evaluate the degree of repair of subchondral bone. 16 weeks after surgery the same evaluations as those of 4 weeks after surgery were performed, and in addition 10 knees in each group were subjected to immunohistochemical and collagen orientation evaluation. The mechanical properties of 6 knees in each group were also evaluated 16 weeks after surgery.

1.4. Macroscopic, Histological and Immunohistochemical Evaluation Methods

4 weeks and 16 weeks after surgery rabbits were euthanized, after which the knee joints including the femoral trochlea were removed, the defects were photographed with a digital camera, and the repaired tissue in each group was macroscopically evaluated by ICRS scoring (n=10) (Brittberg M et al., ICRS Newsletter (1998) 1, 5-8) (Table 2).

TABLE 2
ICRS Scoring

| Degree of defect repair | |
|---|---|
| In level with surrounding cartilage | 4 |
| 75% repair of defect depth | 3 |
| 50% repair of defect depth | 2 |
| 25% repair of defect depth | 1 |
| 0% repair of defect depth | 0 |
| Integration to border zone | |
| Complete integration with surrounding cartilage | 4 |
| Demarcating border <1 mm | 3 |
| ¾ of graft integrated, ¼ with notable border >1 mm width | 2 |
| ½ of graft integrated, ½ with notable border >1 mm width | 1 |
| From no contact to 1/4 of graft integrated with surrounding cartilage | 0 |
| Macroscopic appearance | |
| Intact smooth surface | 4 |
| Fibrillated surface | 3 |
| Small, scattered fissures or cracks | 2 |
| Several small or few but large fissures | 1 |
| Total degeneration of grafted area | 0 |

To use the same specimens for histochemical evaluation, the specimens were fixed with 10% paraformaldehyde, and after decalcification with 10% EDTA, 5 μm-thick paraffin sections were prepared as sagittal sections passing through the center of the defect. The sections were stained with Hematoxylin Eosin (HE) and Safranin-O (Saf-O), and repaired tissue in each group was evaluated histologically by Niederauer scoring (n=10) (Niederauer et al., Biomaterials (2000) 21, 2561-2574) (Table 3).

TABLE 3
Niederauer scoring

| Nature of the predominant tissue | |
|---|---|
| Hyaline cartilage | 4 |
| Mostly hyaline cartilage | 3 |
| Mixed hyaline and fibrocartilage | 2 |
| Mostly fibrocartilage | 1 |
| Some fibrocartilage, mostly nonchondrocytic cells | 0 |
| Structural characteristic | |
| Surface regularity | |
| Smooth and intact | 3 |
| Superficial horizontal lamination | 2 |
| Fissures | 1 |
| Severe disruption, including fibrillation | 0 |
| Structural integrity, homogeneity | |
| Normal | 2 |
| Slight disruption | 1 |
| Severe disintegration, disruption | 0 |
| Thickness | |
| 100% of adjacent cartilage | 2 |
| 50-100% of normal cartilage | 1 |
| 0-50% of normal cartilage | 0 |
| Bonding to adjacent cartilage | |
| Bonded at both ends of graft | 2 |
| Bonded at one end or partially at both ends | 1 |
| Not bonded | 0 |
| Freedom from cellular changes of degeneration | |
| Hypocellularity | |
| Normal cellularity | 2 |
| Slight hypocellularity | 1 |
| Moderate hypocellularity or hypocellularity | 0 |
| Chondrocyte clustering | |
| No clusters | 2 |
| <25% of the cells | 1 |
| 25-100% of the cells | 0 |
| Freedom from degenerative changes in adjacent cartilage | |
| Normal cellularity, no clusters, normal staining | 3 |
| Normal cellularity, mild clusters, moderate staining | 2 |
| Mild or moderate hypo/hypercellularity, slight staining | 1 |
| Severe hypocellularity, poor or no staining | 0 |
| Subchondral bone | |
| Reconstruction of subchondral bone | |
| Normal | 3 |
| Reduced subchondral bone reconstruction | 2 |
| Minimal subchondral bone reconstruction | 1 |
| No subchondral bone reconstruction | 0 |
| Inflammatory response in subchondral bone region | |
| None/mild | 2 |
| Moderate | 1 |
| Severe | 0 |
| Safranin-O staining | |
| Normal or near normal | 3 |
| Moderate | 2 |
| Slight | 1 |
| None | 0 |

In the immunohistological evaluation 16 weeks after surgery, the specimens were stained with type II collagen antibodies (Fuji Pharma Co., Ltd., Toyama, Japan).

1.3. Methods for Evaluating Collagen Orientation With Polarized Light Microscope

16 weeks after surgery, the HE stained sections were observed with a polarized light microscope (PLM), and the collagen orientation of the repaired tissue was evaluated (Roberts et al., 2009; Ross et al., 2013). Collagen orientation is reported to be associated with the mechanical strength of articular cartilage (Rieppo et al., 2003). To confirm the collagen orientation of the repaired cartilage tissue, the mounted tissue sections were rotated by 0°, 45° and 90°, and slight changes were observed. Images taken with a digital camera (DS-5M-L1: Nikon) were each evaluated using a PLM qualitative scoring system (n=10) (Changoor et al., Osteoarthritis Cartilage (2011) 19 (1), 126-135) (Table 4).

TABLE 4
Polarized light microscope (PLM) qualitative scoring

| Score | Description |
|---|---|
| 0 | Evidence of fiber organization, seen as sparse bright patches |
| | throughout the specimen. These patches do not have parallel |
| | alignment at the surface of the specimen nor perpendicular |
| | alignment in the deep zone (DZ), but are randomly oriented in |
| | the specimen. |
| 1 | Birefringent tissue of the expected orientation in the DZ |
| | with fibers oriented mainly perpendicular (±30) to the |
| | cartilage-bone interface and occupying less than ~50% of |
| | the thickness of the noncalcified tissue on average. Little |
| | additional evidence of birefringent tissue is apparent, other |
| | than randomly oriented patches. Birefringent tissue may have |
| | inconsistent thickness and intensity of birefringence across |
| | the lateral direction of the specimen. The specimen texture |
| | may be smooth, patchy, or granular. |
| 2 | Identical to a score of 1 except that the DZ occupies more |
| | than ~50% of the thickness of the noncalcified tissue. |
| | Alternatively, a second region of birefringent tissue may be |
| | present above the DZ that may have any orientation (parallel |
| | to the articular surface, obliquely oriented to the articular |
| | surface, or multiple orientations) except for vertical. In this |
| | case, the DZ may then occupy <50% of the thickness of the |
| | noncalcified tissue. |
| 3 | Zonal organization with birefringent tissue in the DZ |
| | perpendicular to the cartilage-bone interface (±30), and |
| | birefringent tissue at the articular surface that is either |
| | aligned parallel to the surface or that has multiple orientations. |
| | These two zones are separated by a third nonbirefringent region |
| | that is appropriate to the species from which the specimen was |
| | taken; for example, in human articular cartilage, it may be a thin |
| | nonuniform region that is difficult to distinguish compared with |
| | the consistent dark band observed in equine articular cartilage. |
| | Alternatively, the two birefringent zones are separated by a |
| | birefringent region with orientation that is neither parallel nor |
| | perpendicular. Zonal thicknesses are heterogeneous across the |
| | lateral direction of the specimen. The specimen texture may be |
| | smooth, patchy, or granular. |
| 4 | Identical to a score of 3 except that the orientation in the |
| | superficial zone (SZ) must be parallel to the surface and the |
| | transitional zone (TZ) must be appropriate to the species from |
| | which the specimen was taken; for example, in human articular |
| | cartilage, it may be a thin nonuniform region that is difficult |
| | to distinguish compared with the consistent dark band observed in |
| | equine articular cartilage. In addition to these characteristics, |
| | each zone should approximate the zonal proportions for the species |
| | from which the specimen was taken; for example, in human |
| | articular cartilage, the DZ should be the largest, occupying >50% |
| | of the total thickness of noncalcified tissue. The transitional |
| | and SZs are smaller and the TZ may be larger than the SZ. |
| 5 | Displays birefringence patterns of young adult hyaline articular |
| | cartilage with distinct, superficial, and deep zones with uniform |
| | birefringence, indicating parallel and perpendicularly oriented |
| | fibers, respectively, separated by an appropriate TZ. Zonal |
| | thicknesses are appropriate for the species and location from which |
| | the specimen was taken and are relatively homogeneous across the |
| | lateral direction of the specimen. Overall, the specimen |
| | birefringence has a uniform smooth texture and is neither granular |
| | nor patchy. |

1.5. Method for Evaluating Amount of Subchondral Bone Repair by Micro-CT

After the excised knee samples had been evaluated macroscopically as described above, the amount of subchondral bone was measured quantitatively (n=10) by micro-CT (R_mCT2; Rigaku, Tokyo, Japan) 4 weeks and 16 weeks after surgery, and CT images were taken of specimens of each knee sample to evaluate repair of subchondral bone in repaired tissue. The imaging methods and conditions were as described in Baba et al., Tissue Eng Part C Methods (2015) 21 (12), 1263-73; and Hishimura et al., The American Journal of Sports Medicine (2019) e47(2), 468-478. In the resulting images, osteochondral defects of actual prepared defect parts (cylindrical defects 5 mm in diameter and 2 mm deep) were set as the target regions, and the amount of calcified bone in that region was calculated as the amount of subchondral bone repair (bone volume, hereunder BV) (mm³) using Image J software (National Institutes of Health, Bethesda, Md.).

1.6. Method for Measuring Mechanical Properties

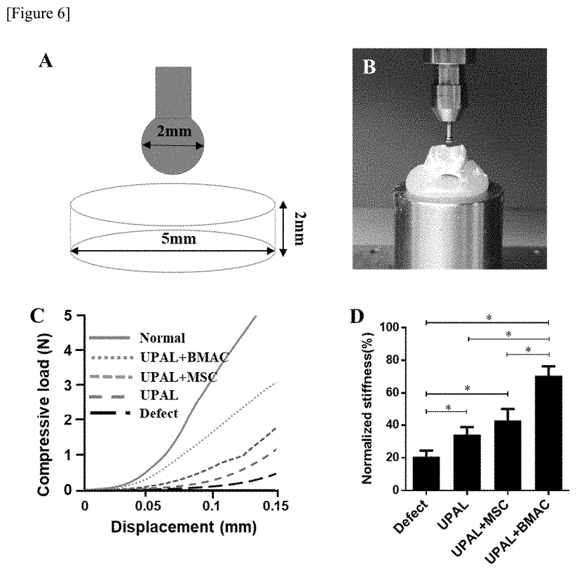

A mechanical tester owned by this research facility (Autograph AG-X, Shimadzu Co., Tokyo, Japan) was used to measure the mechanical properties of the repaired tissue in the defect parts of each knee sample 16 weeks after surgery (Sadia et al., Macromolecules (2016) 49, 5630-5636.). Using a dental room temperature polymerization resin (PMMA) as a base, each knee sample was fixed facing perpendicular to the measuring indenter (hemispherical diameter 2 mm) (Wada et al., Acta Biomater (2016) 15, 44, 125-34), and measurement was performed by pressing the indenter against the measurement site at a rate of 0.5 mm/minute (FIG. 6A). In addition to the center of the repaired tissue, a normal cartilage part near the defect part was also measured as a measurement site to provide a reference value (FIG. 6B). A load-deformation curve was plotted based on the results, and the slope of the part of the curve that approximates the first straight line (reflecting fine deformation of the surface of the measured tissue) was calculated as the stiffness (N/mm). In each sample, the calculated stiffness was converted to a percentage notation based on normal cartilage and studied as normalized stiffness.

1.7. Statistical Analysis

The data were given as average values±standard deviation (SD). Data comparison among four groups was performed by Steel-Dwass test, and between two groups by Mann-Whitney U test. Analysis was performed using JMP Pro 14.1 statistical analysis software (SAS Institute, Cary, N.C.), and the significance level was less than 0.05.

2. Results

### 2.1. Results of Macroscopic Evaluation

No findings indicating post-operative infection were found in the samples in any group after 4 weeks or 16 weeks.

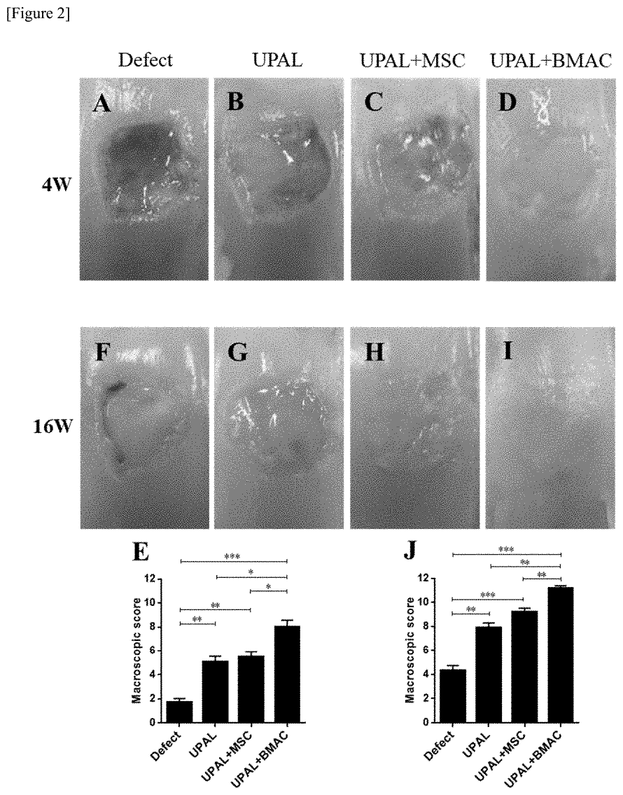

4 weeks after surgery, the defect parts in the Defect group were depressed, with irregular surfaces completely covered with opaque fibrous tissue (FIG. 2A). The defect parts in the UPAL group and UPAL+MSC group were slightly flattened but were also partly covered with opaque tissue and exhibited cracks and small depressions on the surface and the surrounding fused parts (FIGS. 2B, 2C). In the UPAL+BMAC group the defect parts were mostly flattened and completely covered with semi-transparent hyaline cartilage tissue with only slight cracks on the surface (FIG. 2D). In terms of overall macroscopic scoring (ICRS score), the scores in the UPAL+BMAC group (mean±SD: 8.00±1.83) were significantly higher than in the Defect group (1.70±1.06, P<0.01), the UPAL group (5.1±1.45, P<0.05) or the UPAL+MSC group (5.5±1.35, P<0.05) (FIG. 2E).

16 weeks after surgery, the defect parts in the defect group were somewhat flattened but were repaired with opaque tissue exhibiting large cracks and depressions on the surface or the surrounding fused parts (FIG. 2F). The defect parts in the UPAL group and UPAL+MSC group were mostly flattened and completely covered with semi-transparent tissue exhibiting small cracks and depressions on the surface (FIGS. 2G, 2H). The defect parts in the UPAL+BMAC group had generally the same form as the surrounding normal cartilage and were completely covered with smooth-transparent hyaline cartilage tissue on the surface (FIG. 21). In terms of overall macroscopic scoring (ICRS score), in the same way as 4 weeks after surgery, the scores in the UPAL+BMAC group (11.20±0.63) were significantly higher than in the Defect group (4.30±1.42, p<0.01), the UPAL group (7.90±1.29, p<0.01) or the UPAL+MSC group (9.20±1.03, p<0.01) (FIG. 2J).

2.2. Results of Histochemical Evaluation

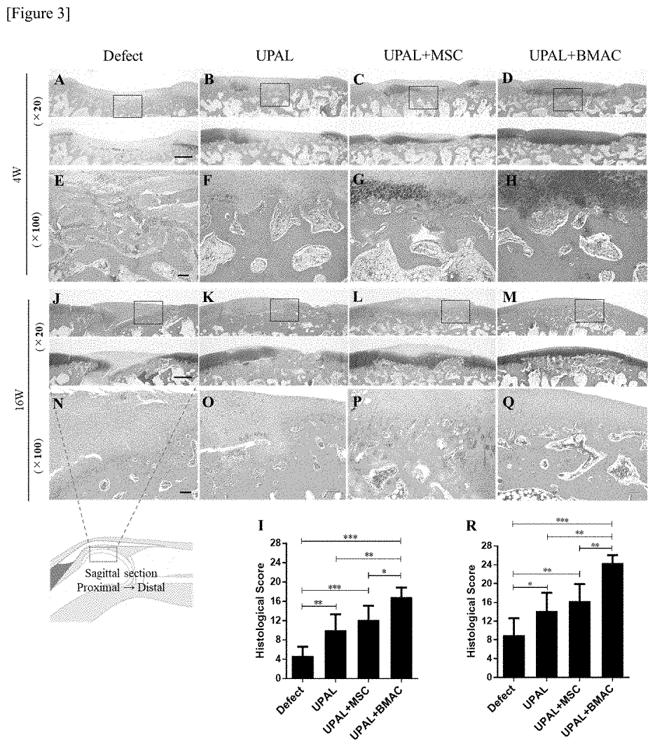

4 weeks after surgery, fibrotic scar tissue was observed in the defect parts in the Defect group (FIG. 3A). Safranin-O-stained hyaline cartilage tissue was observed in parts of the defect parts in the UPAL and UPAL+MSC groups (FIGS. 3B, 3C). On the other hand, favorable hyaline cartilage tissue stained with Safranin-O was observed to be somewhat thicker than the surrounding tissue in the defect parts of the UPAL+BMAC group (FIG. 3D). In the enlarged images (×100), while moderate inflammatory cell infiltration was observed in the repaired tissue in the Defect group, almost no inflammatory findings were seen in the UPAL, UPAL+MSC and UPAL+BMAC groups (FIGS. 3E to FIG. 3H). In terms of Niederauer scores, the score in the UPAL-BMAC group (16.70±1.83) was significantly higher than in the Defect group (4.70±1.89, P<0.001), the UPAL group (10.0±3.33, P<0.01) or the UPAL+MSC group (12.2±2.90, P<0.05) (FIG. 31).

16 weeks after surgery, the amount of hyaline cartilage tissue in the repaired tissue in the Defect group was greater than 4 weeks after surgery, but over half was fibrous tissue not stained by Safranin-O, and the normal cartilage parts surrounding the defects had reduced Safranin-O staining, suggesting degeneration (FIG. 3J). Half to more than half of the repaired tissue in the UPAL and UPAL+MSC groups was occupied by hyaline cartilage tissue stained with Safranin-O (FIGS. 3K, 3L). By contrast, in the UPAL+BMAC group the defects were covered with strongly Safranin-O-stained hyaline cartilage tissue having roughly the same consistency and properties as the surrounding normal cartilage (FIG. 3M). In the enlarged images (×100), no obvious inflammatory findings were seen in any group. While formation of moderate to mild high-density spindle-shaped cells and cell clusters was observed in the repaired tissue in the Defect, UPAL and UPAL+MSC groups, in the UPAL+BMAC group the area was filled with normal-density round cells, and almost no cell clusters were observed. Looking at subchondral bone repair, almost none was seen in the Defect group, while the UPAL and UPAL+MSC groups exhibited incomplete subchondral bone repair and highly irregular cartilage/subchondral bone interfaces, but in the UPAL+BMAC group the subchondral bone repair was broad and the cartilage/subchondral bone interfaces were regular (FIGS. 3N to 3Q). The Niederauer scores were also significantly higher in the UPAL+BMAC group (24.40±1.65) than in the Defect group (9.00±3.65, P<0.001), the UPAL group (14.20±3.88, P<0.01) or the UPAL+MSC group (16.30±3.62, P<0.01) (FIG. 3R). Table 5 shows the histochemical score results broken down by category.

TABLE 5
Average histochemical scores

| Histological score | 4 weeks | | | | 16 weeks | | | |
|---|---|---|---|---|---|---|---|---|
| by Niederauer | Defect | UPAL | U + M | U + B | Defect | UPAL | U + M | U + B |
| Nature of the | 0.3 | 1.3 | 1.7 ^{a} | 2.7 ^{a.b.c} | 1.0 | 1.2 | 1.7 | 3.3 ^{a.b.c} |
| predominant tissue | | | | | | | | |
| Surface regularity | 0.6 | 1.4 | 1.8 ^{a} | 2.0 ^{a.b} | 1.4 | 1.9 | 2.1 ^{a} | 2.4 ^{a} |
| Structural integrity, | 0.0 | 0.6 ^{a} | 0.6 ^{a} | 1.3 ^{a} | 0.3 | 0.7 | 0.8 | 1.7 ^{a.b.c} |
| homogeneity | | | | | | | | |
| Thickness | 0.2 | 0.6 | 1.0 | 1.7 ^{a} | 0.4 | 1.0 | 1.0 | 1.9 ^{a.b.c} |
| Bonding to adjacent | 0.0 | 0.4 | 0.2 | 1.0 ^{a.c} | 0.4 | 0.8 | 1.1 | 1.8 ^{a.b.c} |
| cartilage | | | | | | | | |
| Hypocellularity | 0.0 | 0.0 | 0.0 | 0.0 | 0.3 | 1.0 | 1.1 ^{a} | 2.0 ^{a.b.c} |
| Chondrocyte clustering | 0.0 | 0.0 | 0.2 | 0.0 | 0.2 | 0.8 ^{a} | 0.9 ^{a} | 1.2 ^{a} |
| Adjacent cartilage | 1..6 | 1.8 | 1.9 | 1.9 | 1.4 | 1.9 | 2.0 | 3.0 ^{a.b.c} |
| degeneration | | | | | | | | |
| Reconstruction of | 0.7 | 0.6 | 1.4 ^{b} | 1.6 ^{a.b} | 0.9 | 1.7 | 2.1 ^{a} | 2.7 ^{a.b} |
| subchondral bone | | | | | | | | |
| Inflammatory response | 1.0 | 1.7 | 1.7 | 1.9 ^{a} | 1.9 | 2.0 | 2.0 | 2.0 |
| Safranin-O staining | 0.3 | 1.2 | 1.7 ^{a} | 2.7 ^{a.b.c} | 0.8 | 1.2 | 1.5 | 2.4 ^{a.b.c} |
| Total score | 4.7 | 10.0 ^{a} | 12.2 ^{a} | 16.9 ^{a.b.c} | 9.0 | 14.2 ^{a} | 16.3 ^{a} | 24.4 ^{a.b.c} |

UPAL + MSC: U + M; UPAL + BMAC: U + B
^{a} P < 0.05 vs Defect group at the same time.
^{b} P < 0.05 vs UPAL group at the same time.
^{c} P < 0.05 vs UPAL + MSC group at the same time.

2.3. Collagen Orientation Evaluation Results

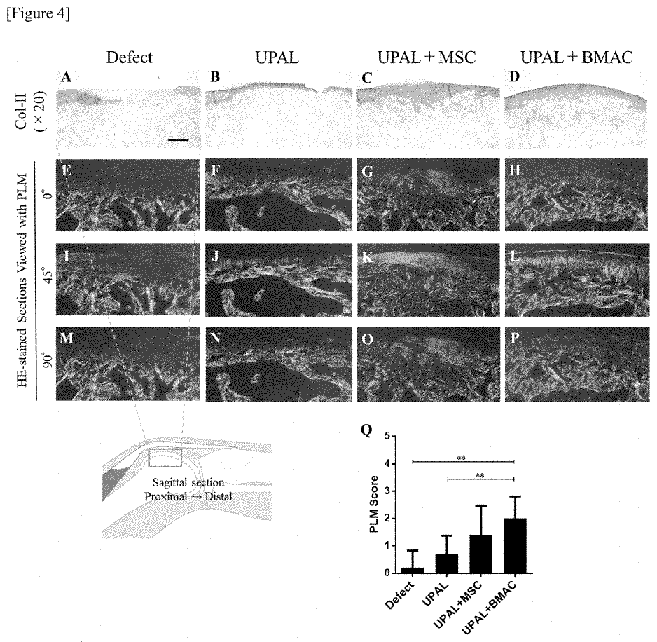

16 weeks after surgery, repaired tissue in each group was evaluated using the PLM qualitative scoring described above. Almost no vertical alignment in the deep layer or other 2-layer alignment was observed in the Defect group (FIGS. 4E, 4I, 4M). Partial vertical alignment was observed in the UPAL and UPAL+MSC groups, and the other two layers also appeared slightly, but the thicknesses of the layers were irregular (FIGS. 4F, 4J and 4N and 4G, 4K and 4O). In the UPAL+BMAC group, on the other hand, vertical alignment of the deep layer and transitional layer was observed along with horizontal alignment of the surface layer, with all layers being of somewhat regular thickness (FIGS. 4H, 4L, 4P). In terms of scoring, the scores in the UPAL+BMAC group (2.00±0.82) were significantly higher than in the Defect group (0.20±0.63, P<0.01) and the UPAL group (0.70±0.67, P<0.01), but the difference with the UPAL+MSC group was not significant (1.40±1.07, P=0.73) (FIG. 4Q).

2.4. Results for Amount of Subchondral Bone Repair

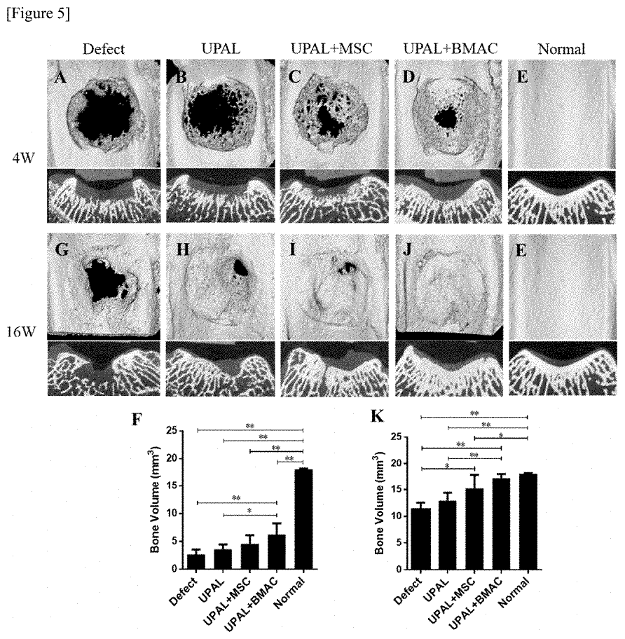

4 weeks after surgery, no repair of subchondral bone was seen in the Defect group, while slight or partial repair was seen in the UPAL and UPAL+MSC groups. In the UPAL+BMAC group there was large-scale repair with only small cracks remaining (FIGS. 5A to 5D). When the amount of repair to subchondral bone was measured quantitatively by Micro-CT, the amount in the UPAL+BMAC group (6.28±1.98 mm³) was significantly greater than in the Defect group (2.68±0.89 mm³, P<0.01) or the UPAL group (3.59±0.88 mm³, P<0.05), but the difference with the UPAL+MSC group (4.59±1.56 mm³, P=0.387) was not significant. In all groups, the amount of subchondral bone repair was significantly different from the normal group (FIG. 5E) (Normal group: 18.17±0.17 mm³, all groups P<0.01) (FIG. 5F).

16 weeks after surgery, repair to subchondral bone in the Defect and UPAL groups consisted of partial repair with large defects or moderate defects remaining. The subchondral bone in the UPAL+MSC group was repaired to a degree that left small cracks. By contrast, the subchondral bone in the UPAL+BMAC group was largely repaired, with reconstructed bone structure showing favorable continuity with the surrounding subchondral bone, suggesting that bone fusion had occurred. No excessive bone formation was seen in any of the specimens (FIGS. 5G to 5J). In quantitative measurement of subchondral bone repair, the amount was significantly greater in the UPAL+BMAC group (17.28±0.77 mm³) than in the Defect group (11.37±1.20 mm³, P<0.01) and the UPAL group (12.92±1.50 mm³, P<0.01) as it was after 4 weeks. There was a tendency towards greater bone volume in comparison with the UPAL+MSC group (15.24±2.67 mm³, P=0.579), but the difference between the groups was not obviously significant. As in the case of the 4-week results, the amounts were significantly smaller in the Defect group (P<0.01), the UPAL group (P<0.01) and the UPAL+MSC group (P<0.05) in comparison with the normal group (18.17±0.17 mm³), but the significant difference between the normal group and the UPAL+BMAC group (17.28±0.77 mm³, P=0.12) had been eliminated (FIG. 5K).

2.5. Results of Mechanical Properties Evaluation

16 weeks after surgery, a mechanical test was performed with an indentation tester to evaluate the mechanical properties of the repaired tissue. A typical press-fit curve is shown for repaired tissue in each group (FIG. 6C). The stiffness (N/mm) in each case was calculated from the region (0-0.08 mm) where the slope is initially constant in this curve, and is normalized as described above (Defect group: R²=0.986±0.018, UPAL group: R²=0.995±0.004, UPAL+MSC group: R²=0.996±0.001, UPAL+BMAC group: R²=0.987 ±0.018). The normalized stiffness values (%) were significantly higher in the UPAL+BMAC group (70.69%±5.75%) in comparison with the Defect group (20.70%±3.71%, P<0.05), the UPAL group (34.19%±4.63%, P<0.05) and the UPAL+MSC group (42.89%±7.43%, P<0.05) (FIG. 6D).

3. Discussion

From a macroscopic, histological and chemical perspective, tissue repair was significantly better in the combined group using UPAL gel and BMAC than in the UPAL single group and the combined UPAL gel and MSC group. These results suggest that the synergistic effects of UPAL gel and BMAC promote better repair of hyaline cartilage and subchondral bone when the two are transplanted together. In particular, the subchondral bone volume 16 weeks after surgery in the UPAL gel and BMAC combined group exhibited repair to a degree that there was no difference with the normal group. It is generally considered difficult to obtain therapeutic results with subchondral bone, so such an effect is unexpected.

These results suggest the possibility of a favorable therapeutic effect against large-sized osteochondral defects from combined transplantation therapy with UPAL gel and BMAC.

Combined use of UPAL gel (a biomaterial that can be injected locally) with concentrated bone marrow aspirate can be an effective one-stage therapy for large articular cartilage or osteochondral injury. This therapy could also be a new treatment method enabling one-stage arthroscopic surgery in place of autologous chondrocyte implantation (ACI) and autologous osteochondral column transplantation (AOT), which, are the principal therapies for severe cartilage or osteochondral defects in Japan.

1.-18. (canceled)

19. A method for treating cartilage injury lesion in a subject in need thereof comprising applying (i) a composition comprising a monovalent metal salt of alginic acid and (ii) a concentrated bone marrow aspirate to a cartilage injury lesion of the subject, wherein the composition has fluidity when applied to the cartilage injury lesion.

20. The method according to claim 19, wherein the concentrated bone marrow aspirate is derived from the subject.

21. The method according to claim 19, wherein the application comprises either (a) or (b) below:
(a) the concentrated bone marrow aspirate is mixed with the composition comprising the monovalent metal salt of alginic acid, and the resulting mixture is applied to the cartilage injury lesion of the subject, or
(b) the concentrated bone marrow aspirate and the composition comprising the monovalent metal salt of alginic acid are each applied to the cartilage injury lesion of the subject rather than being mixed together before application to the cartilage injury lesion.

22. The method according to claim 19, wherein a number of bone marrow mesenchymal stem cells in the concentrated bone marrow aspirate is from 1×10² to 1×10⁷ cells/mL.

23. The method according to claim 19, wherein the cartilage injury is accompanied by damage to the subchondral bone.

24. The method according to claim 19, wherein the cartilage injury lesion is at least 4 cm² in size.

25. The method according to claim 19, wherein the cartilage injury lesion is articular cartilage.

26. The method according to claim 19, wherein an apparent viscosity of the composition having fluidity before being combined with the concentrated bone marrow aspirate is from 500 mPa·s to 10,000 mPa·s as measured with a cone plate viscometer (sensor 35/1) with a measurement temperature of 20° C. for the measurement, a rotational speed of 0.5 rpm and a reading time of 2.5 minutes and using an average of values taken during a period from 0.5 to 2.5 minutes after start of measurement.

27. The method according to claim 19, wherein the monovalent metal salt of alginic acid has a weight-average molecular weight (absolute molecular weight) of at least 80,000 as measured by a GPC-MALS method.

28. The method according to claim 19, wherein a concentration of the monovalent metal salt of alginic acid in the composition before being combined with the concentrated bone marrow aspirate is from 0.5 w/w % to 5 w/w %.

29. The method according to claim 19, wherein a curing agent is brought into contact with at least part of a surface of the composition after the composition is applied to the cartilage injury lesion.

30. The method according to claim 19, wherein the composition does not contain the curing agent in an amount that cures the composition before being applied to the cartilage injury lesion of the subject.

31. The method according to claim 29, wherein the curing agent is a divalent or higher metal ion compound.

32. The method according to claim 19, wherein the monovalent metal salt of alginic acid is a low-endotoxin monovalent metal salt of alginic acid.

33. The method according to claim 19, wherein the cartilage injury lesion is associated with a cartilage-related disease.

34. The method according to claim 33, wherein the cartilage-related disease is at least one cartilage-related disease selected from the group consisting of osteoarthritis, traumatic cartilage defect, peripheral traumatic cartilage defect, traumatic cartilage injury, osteonecrosis, osteochondritis dissecans, subchondral bone defect, degeneration of cartilage and/or subchondral bone, and misalignment in joints.

35. The method according to claim 19, further comprising performing at least one additional treatment on the subject selected from the group consisting of repairing cartilage tissue and/or subchondral bone, regenerating cartilage tissue and/or subchondral bone, suppressing progress of degeneration in cartilage tissue and/or subchondral bone, regenerating hyaline cartilage, alleviating pain, alleviating postoperative pain, reducing dysfunction, alleviating clinical symptoms, and preventing or suppressing the recurrence of cartilage-related disease.
